(19) United States
(12) Patent Application Publication
(10) Pub. No.: US 2024/0218337 A1
(43) Pub. Date: Jul. 4, 2024

(54) FUSION PROTEIN TARGETING PD-L1 AND NEUTRALIZING GAS6 AND USES THEREOF

(71) Applicant: DEVELOPMENT CENTER FOR BIOTECHNOLOGY, TAIPEI CITY (TW)

(72) Inventors: CHU-BIN LIAO, TAIPEI CITY (TW); HUI-CHIEH YU, TAIPEI CITY (TW); HSIEN-YU TSAI, TAIPEI CITY (TW); WEN-BIN KE, TAIPEI CITY (TW); MENG-TING CAI, TAIPEI CITY (TW)

(73) Assignee: DEVELOPMENT CENTER FOR BIOTECHNOLOGY, TAIPEI CITY (TW)

(21) Appl. No.: 18/396,245

(22) Filed: Dec. 26, 2023

Related U.S. Application Data
(60) Provisional application No. 63/435,827, filed on Dec. 29, 2022.

(51) Int. Cl. C12N 9/12; A61K 38/00; A61P 35/00; C07K 16/28; G01N 33/574; G01N 33/68

(52) U.S. Cl. CPC C12N 9/12; A61P 35/00; C07K 16/2827; G01N 33/57484; G01N 33/6857; A61K 38/00; C07K 2317/52; C07K 2317/565; C07K 2319/33; C12Y 207/10001; G01N 2333/70503

(57) ABSTRACT

The present disclosure relates to a fusion protein comprising a Gas6 binding portion and an antigen binding portion comprising an anti-PD-L1 antibody or antigen-binding fragment thereof. The present disclosure also relates to a method for treating and/or detecting a cancer in a subject in need, and a kit for detecting a cancer in a sample.

### REFERENCE TO SEQUENCE LISTING SUBMITTED VIA EFS-WEB

This application includes an electronically submitted sequence listing in .xml format. The .xml file contains a sequence listing entitled “US14168.xml” created on Dec. 26, 2023 and is 107,000 bytes in size. The sequence listing contained in this .xml file is part of the specification and is hereby incorporated by reference herein in its entirety.

### FIELD OF THE INVENTION

The present disclosure relates to a novel fusion protein, particularly to a fusion protein targeting PD-L1 (programmed death-ligand 1) and neutralizing Gas 6 (growth arrest-specific 6).

### BACKGROUND OF THE INVENTION

Cancer manipulates the tumor microenvironment for its own sake. Cancer can elicit an immunosuppressive microenvironment to benefit its own development, progression, metastasis, and therapeutic resistance. Immune checkpoint blockers have emerged as a promising cancer immunotherapy for decades. By harnessing the tumor microenvironment of patients with immune checkpoint blockers, 15 to 25 percent of patients bearing different cancers have shown positive results. The overall survival of immune checkpoint blockers in patients even surpasses that of standard treatments for those who respond distinguishingly well.

However, the limitations of monotherapy with PD-L1/PD-1 blockades make combination treatment strategies necessary to broaden their applications, enhance their efficacy, and reduce their toxicities. Most disappointed, combination therapies based on immunotherapies with approved standard therapies, though partially effective, are restricted by the occurrence of severe adverse effects. Thus, there is need for developing a novel approach to treating cancer.

### SUMMARY OF THE INVENTION

Embodiments of the disclosure relate to tumor treatment or diagnoses via a fusion protein as a fusion protein including (i) a Gas6 binding portion. Embodiments of the disclosure relate to uses of such molecules (e.g., for treating cancer) and (ii) an antibody, or antigen-binding fragment thereof, that binds to an immune checkpoint protein, such as programmed death ligand 1 (PD-L1), and methods of making such molecules.

Accordingly, the present disclosure provides a fusion protein comprising
a Gas6 binding portion; and an antigen binding portion comprising an antibody or antigen-binding fragment thereof that is specific to an epitope in PD-L1.

In some embodiments of the present disclosure, the Gas6 binding portion comprises an extracellular domain of RTK (receptor tyrosine kinase) of TAM family (Tyro-3, Axl, MerTK).

In some embodiments of the present disclosure, the antibody or antigen-binding fragment thereof comprises complementarity determining regions (CDRs) of a heavy chain variable region (VH) and complementarity determining regions of a light chain variable region (VL), wherein the complementarity determining regions of the heavy chain variable region comprise VH-CDR1, VH-CDR2 and VH-CDR3, and the complementarity determining regions of the light chain variable region comprise VL-CDR1, VL-CDR2 and VL-CDR3.

Examples of the antibody or antigen-binding fragment thereof include, but are not limited to an antibodies (a) to (k) or antigen-binding fragment thereof, 3G10 of U.S. Pat. No. 7,943,743, 12A4 of U.S. Pat. No. 7,943,743, 10A5 of U.S. Pat. No. 7,943,743, 5F8 of U.S. Pat. No. 7,943,743, 10H10 of U.S. Pat. No. 7,943,743, 1B12 of U.S. Pat. No. 7,943,743, 7H1 of U.S. Pat. No. 7,943,743, 11E6 of U.S. Pat. No. 7,943,743, 12B7 of U.S. Pat. No. 7,943,743, 13G4 of U.S. Pat. No. 7,943,743, MDX-1105, MEDI-4736, atezolizumab, durvalumab, avelumab, MDX-1105, envafolimab, cosibelimab, CK-301, CS-1001, SHR-1316, CBT-502, or BGB-A333.

In some embodiments of the present disclosure, the antibody or antigen-binding fragment thereof comprises a heavy chain constant region and a light chain constant region. In one embodiment of the disclosure, the constant region contains a mutation at an amino acid position corresponding to N297 of IgG1.

In some embodiments of the present disclosure, the antigen binding portion comprises an Fab fragment, an F(ab′)₂ fragment, an ScFv fragment, a chimeric antibody, or a nanobody.

In some embodiments of the present disclosure, the antigen binding portion is multi-specific.

In some embodiments of the present disclosure, the Gas6 binding portion is fused to the antigen binding portion through a peptide linker.

In some embodiments of the present disclosure, the Gas6 binding portion is fused to a heavy chain of the antigen binding portion.

In some embodiments of the present disclosure, the Gas6 binding portion is fused to the C-terminus of a heavy chain of the antigen binding portion.

The present disclosure further provides a pharmaceutical composition, including:
an effective amount of the fusion protein as disclosed herein or the genetically engineered cell as disclosed herein; and
a pharmaceutically acceptable carrier.

The present disclosure further provides a method for treating, prophylactic treating and/or preventing a cancer in a subject in need thereof, comprising administering the subject with an effective amount of the fusion protein as disclosed herein.

The present disclosure further provides a method for detecting a cancer in a subject in need thereof, comprising contacting a sample derived from the subject with the fusion protein as disclosed herein.

The present disclosure further provides a kit for detecting a cancer in a sample, comprising the fusion protein as disclosed herein.

Examples of the cancer include, but are not limited to bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer, and thyroid cancer.

The present disclosure further provides a method for detecting PD-L1 in a sample, comprising contacting the sample with the fusion protein as disclosed herein.

The present disclosure further provides a method for neutralizing Gas6 in a sample, comprising contacting the sample with the fusion protein as disclosed herein.

### BRIEF DESCRIPTION OF DRAWINGS

FIG. 1 shows the favorable spatial conformations of the disclosure.

FIG. 2 shows that example_1 and example_2 can efficiently trap human Gas6, therefore achieve cell growth inhibition in Axl-overexpressing Ba/F3 cell line.

FIG. 3 shows that example_3, example_4, example_5, and example_6 can efficiently trap human Gas6, therefore achieve cell growth inhibition in Axl-overexpressing Ba/F3 cell line.

FIG. 4 shows that example_7, example_8 and example_9 can efficiently trap human Gas6, therefore achieve cell growth inhibition in Axl-overexpressing Ba/F3 cell line.

FIG. 5 shows that the in vivo tumor growth inhibition function of example_2 surpasses that of atezolizumab, homemade Axl-Fc, and combination therapy.

FIG. 6 shows that mice which were intraperitoneally injected molecules containing the Gas6 binding portion moiety demonstrated decreased serum Gas6 concentration.

FIG. 7 shows that mice which achieved complete remission in the example_2 group demonstrated better immunological memory than that in the atezolizumab group while being rechallenged. Wherein, 1 out of 6 mice in the atezolizumab group and 2 out of 6 mice in the example_2 group achieved complete remission.

FIG. 8 shows the impressive in vivo anti-tumor efficacy of example_3.

### DETAILED DESCRIPTION OF THE INVENTION

Unless otherwise defined, scientific and technical terms used herein shall have the meanings that are commonly understood by those of ordinary skill in the art. Furthermore, unless otherwise required by context, singular terms shall include pluralities and plural terms shall include the singular. Generally, nomenclatures used in connection with, and techniques of, cell and tissue culture, molecular biology, and protein and oligo- or polynucleotide chemistry and hybridization described herein are those well-known and commonly used in the art.

The practice of the present disclosure may employ technologies comprising conventional techniques of cell biology, cell culture, antibody technology, and genetic engineering, which are within the ordinary skills of the art. Such techniques are explained fully in the literature.

As utilized in accordance with the present disclosure, the following terms, unless otherwise indicated, shall be understood to have the following meanings: The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually herein.

It must be noted that, as used in this specification and the appended claims, the singular forms “a.” “an,” and “the” include plural referents unless the content clearly dictates otherwise.

The term “antibody”, as used herein, means any antigen-binding molecule or molecular complex comprising at least one CDR that specifically binds to or interacts with a particular antigen (e.g., PD-L1). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (VH). In some embodiments, the heavy chain further comprises a heavy chain constant region. The heavy chain constant region comprises three domains, C_{H1}, C_{H2} and C_{H3}. Each light chain comprises a light chain variable region (VL). In some embodiments, the light chain further comprises a light chain constant region. The light chain constant region comprises one domain (C_{L1}). The VH and VL can be further subdivided into regions of hypervariability, termed CDRs, interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-PD-L1 antibody (or antigen-binding fragments thereof) may be identical to the human germline sequences, or may be naturally or artificially modified. An amino acid consensus sequence may be defined based on a side-by-side analysis of two or more CDRs.

As used herein, the term “being specific to” or “binding specifically to” means that an antibody does not cross react to a significant extent with other epitopes.

As used herein, the term “epitope” refers to the site on the antigen to which an antibody binds.

As used herein, the term “complementarity determining region (CDR)” refers to the non-contiguous antigen combining sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991); by Chothia et al., J. Mol. Biol. 196:901-917 (1987); and MacCallum et al., J. Mol. Biol. 262:732-745 (1996), where the definitions include overlapping or subsets of amino acid residues when compared against each other.

The term “chimeric” antibody as used herein refers to an antibody having variable sequences derived from a non-human immunoglobulin and human immunoglobulin constant regions, typically chosen from a human immunoglobulin template.

As used herein, the term “nanobody” refers to an antibody comprising the small single variable domain (VHH of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals.

The term “antigen-binding fragment” of an antibody, and the like, as used herein, include any naturally occurring, enzymatically obtainable, synthetic, or genetically engineered polypeptide or glycoprotein that specifically binds an antigen to form a complex.

As applied to polypeptides, the term “substantial similarity” or “substantially similar” means that a protein sequences, when optimally aligned with another (reference) protein sequence, such as by the programs GAP or BESTFIT using default gap weights, there is sequence identity in at least 81%, 82%, 83%, 84%, 85%, 86%, 87%, 88%, 89%, 90%, 91%, 92%, 93%, 94%, 95%, 96%, 97%, 98%, 99%, or 100% of amino acid residues, to the entire sequence of said reference protein sequence. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutant thereof. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

As used in the present disclosure, the term “pharmaceutical composition” means a mixture containing a therapeutic agent administered to a mammal, for example a human, for preventing, treating, or eliminating a particular disease or pathological condition that the mammal suffers.

As used herein, the term “therapeutically effective amount” or “effective amount” refers to the amount of an antibody that, when administered to a mammal or other subject for treating a disease, is sufficient to effect such treatment for the disease.

As used herein, the terms “treatment,” “treating,” and the like, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

As interchangeably used herein, the terms “individual,” “subject.” “host.” and “patient.” refer to a mammal, including, but not limited to, murines (rats, mice), non-human primates, humans, canines, felines, ungulates (e.g., equines, bovines, ovines, porcines, caprines), etc.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

“Cancer,” “tumor,” and like terms include precancerous, neoplastic, transformed, and cancerous cells, and can refer to a solid tumor, or a non-solid cancer (see, e.g., Edge et al. AJCC Cancer Staging Manual (7th ed. 2009); Cibas and Ducatman Cytology: Diagnostic principles and clinical correlates (3rd ed. 2009)). Cancer includes both benign and malignant neoplasms (abnormal growth). “Transformation” refers to spontaneous or induced phenotypic changes, e.g., immortalization of cells, morphological changes, aberrant cell growth, reduced contact inhibition and anchorage, and/or malignancy (see, Freshney, Culture of Animal Cells a Manual of Basic Technique (3rd ed. 1994)). Although transformation can arise from infection with a transforming virus and incorporation of new genomic DNA, or uptake of exogenous DNA, it can also arise spontaneously or following exposure to a carcinogen.

As used herein, the term “sample” encompasses a variety of sample types obtained from an individual, subject or patient and can be used in a diagnostic or monitoring assay. The definition encompasses blood and other liquid samples of biological origin, solid tissue samples such as a biopsy specimen or tissue cultures or cells derived therefrom and the progeny thereof.

### Fusion Protein:

The present disclosure provides a fusion protein that targets both PD-L1 and Gas6. The anti-PD-L1 antibody or PD-L1-binding fragment as disclosed herein is fused to portion of Gas6 binding portion (e.g. Axl decoy receptor) which specifically binds to Gas6 and function as a trap for Gas6. Experimental results show that the bifunctional structures of the fusion protein comprising an anti-PD-L1 antibody or PD-L1 binding fragment fused with a Gas6 binding portion (also noted as anti-PD-L1×Gas6 binding portion) disturb neither part of the entity (anti-PD-L1 or Gas6 binding portion) in its binding affinity and blocking activity to its corresponding target. The anti-PD-L1×Gas6 binding portion motif can concentrate to the targeted tumor better than the individual agents. Based on the differences of binding affinity between anti-PD-L1 antibody or PD-L1 binding fragment and Gas6 binding portion to their corresponding targets, anti-PD-L1 antibody or PD-L1 binding fragment dominates the tumor-targeting effects of the anti-PD-L1×Gas6 binding portion. In addition, the anti-PD-L1×Gas6 binding portion unexpectedly demonstrates synergistic effects in cancer treatment as compared to the effect of administering the two agents separately.

Particularly; the present disclosure provides a fusion protein comprising
a Gas6 binding portion; and
an antigen binding portion comprising an antibody or antigen-binding fragment thereof that is specific to an epitope in PD-L1.

The fusion protein can deliver therapeutic benefits to a subject. The fusion protein as disclosed herein, can be used as therapeutics for treating and/or diagnosing a cancer, which are more fully described herein.

One of the inhibitory checkpoint receptors, programmed cell death protein-1 (PD-1), and its ligand, PD-L1, are responsible for exhausting T cells in various tumors. Monoclonal antibodies which counteract PD-L1/PD-1 interactions therefore can stimulate immune cells to perform anticancer responses. PD-L1/PD-1 blockades as monotherapeutics are proved to benefit patients bearing certain cancers with characteristics such as high mutation burden, and upregulated PD-L1 expression. However, the high response rate of PD-L1/PD-1 blockades is limited to patients with certain cancer types. Some patients who are expected to respond to PD-L1/PD-1 blockades based on available biomarkers further puzzle the prediction with negative outcomes. Also, despite the relatively mild adverse effects PD-L1/PD-1 blockades may generally cause, around 10% of patients with PD-L1/PD-1 blockades result in grade 3 to 4 toxicities. The limitations of monotherapy with PD-L1/PD-1 blockades make combination treatment strategies necessary to broaden their disclosures, enhance their efficacy, and reduce their toxicities. Most disappointed, combination therapies based on immunotherapies with approved standard therapies, though partially effective, are restricted by the occurrence of severe adverse effects. Hence, the development of combination therapies with higher survival benefits and fewer adverse effects is still desired.

Growth arrest-specific 6, a member of the vitamin K-dependent family of proteins, is expressed by cancer cells as well as tumor-infiltrating leukocytes. By binding to its receptors, the TAM family of RTKs, Gas6 regulates a diverse array of cellular functions, including cellular proliferation, migration, survival, angiogenesis, and metabolism. RTKs are cell surface receptors that have the capacity to catalyze the phosphorylation of tyrosine residues on target proteins. The TAM family of RTKs consists of Axl, Tyro, and MerTK. All TAM family receptors have an extracellular ligand binding domain, a single-pass transmembrane domain, and intracellular kinase domain, and a tyrosine containing C-terminal tail. Numerous studies have shown that upregulation of Gas6/TAM can promote the development of several cancers and is involved in cancer therapy resistance. Clinically, expression of Gas6 and TAM receptors always predicts poor prognosis. In addition. Gas6 aids tumors in immune evasion by binding to TAM receptors on lingering immune cells.

Gas6 binds the TAM receptors with different affinity, with Axl the highest among the others. Axl is overexpressed by host cells lingering in tumor microenvironment, including several immune cell types, fibroblasts, osteoclasts, and endothelial cells, and is found meant for immune evasion. The elevated expression of Axl receptor tyrosine kinase in various tumors is correlated with poor overall survival in patients. Intrinsic Axl activation made by tumor and extrinsic Axl activation made by recruited suppressive immune cells in tumor microenvironment contribute to the prosperity of cancer. Both by intrinsic and extrinsic effects, the autocrine and paracrine Gas6/Axl signaling axis promote tumor growth, metastasis, immune evasion, and therapeutic resistance in cancer patients. Moreover, Gas6/Axl pathway has also been reported to drive the expression of PD-L1 in cancer cells to prevent T cell activation.

As noted above, avoiding Gas6 from binding to TAM has emerged as an important approach for cancer therapy. Inhibiting PD-L1/PD-1 and Gas6/TAM pathways simultaneously becomes a plausible solution to the limitations of monotherapy with PD-L1 blockades.

The fusion proteins in this disclosure permit localized reduction in TAM kinase activation intrinsically and extrinsically in a tumor by capturing the Gas6 with a decoy receptor, Gas6 binding portion, fused to an antibody moiety, anti-PD-L1, targeting the extracelluar domain of an immune checkpoint protein found on cancer cells and immune cells. This fusion protein, sometimes referred to in this disclosure as anti-PD-L1×Gas6 binding portion, surpasses currently available anti-cancer strategies including but not limited to administration of the antibody and the receptor as separate molecules or in combination because of various reasons. Firstly, the antibody moiety precisely directs the fusion protein to the tumor microenvironment where concentrated Gas6 functions predominantly through autocrine and paracrine routes to increase tumor growth, metastasis, and immune evasion. The resulting advantage (over, for example, administration of the antibody and the receptor as separate molecules) is partly because cytokines function predominantly in the local environment through autocrine and paracrine functions. The antibody moiety directs the cytokine trap to the tumor microenvironment where it can be most effective, by neutralizing the local immunosuppressive autocrine or paracrine effects. Secondly, the fusion protein can tackle tumor growth, metastasis, and immune evasion with the decoy receptor, and revive the local immunosurveillance with the antibody moiety simultaneously. Moreover, the positive feedback of PD-L1 production due to the activation of Gas6/Axl axis also foresees the synergistic effects of this fusion protein. Therefore, broader indications can be applied to with this fusion protein because of the two mechanisms in one entity design. Finally, as a single entity, lower dosage can be used to achieve multifunction with this fusion protein. Armed with multifunction in one entity also expects tolerance to resistance. Furthermore, different spatial arrangements of the fusion protein have been screened by the present inventor. Selected conformations shown in this disclosure are with the Gas6 binding portion on the C-terminal of the antibody moiety where the stop codons-lacking antibody moiety is trailed by the stop codons-containing Gas6 binding portion.

Overall, the disclosure revolutionizes anti-PD-L1-based immunotherapies in three aspects: firstly, the disclosure is precisely driven to tumor to elicit a synergistic anti-tumor effect attributed to the simultaneous blockade of the PD-L1/PD-1 and Gas6/TAM pathway in tumor and its microenvironment. Therefore, the fusion protein enhances the efficacy of anti-PD-L1-based immunotherapies and permits broader indications of anti-PD-L1-based immunotherapies by introducing a fused Gas6 binding portion to the entity; secondly, the fusion protein restricted in tumor and its microenvironment performs multifunction in one entity, achieving maximum effects more specifically with minimum dosage. The multifunction in one entity formats also enable their enhanced tolerance to resistance; the last, the favorable spatial combinations of this fusion protein has been chosen from a variety of candidates in possible spatial combinations. The fusion proteins disclosed in this disclosure thus are also practical results of in silico predictions, helping speed up future innovation of multifunctional proteins comprising an antibody moiety fused to a decoy receptor.

### Gas6 Binding Portion:

Gas6 belongs structurally to the family of plasma vitamin K-dependent proteins. Gas6 has growth factor-like properties through its interaction with receptor tyrosine kinases of the TAM family. Human Gas6 is a 678 amino acid protein that consists of a vitamin K-dependent-carboxyglutamate (Gla)-rich domain that mediates binding to phospholipid membranes, four epidermal growth factor-like domains, and two laminin G-like (LG) domains that mediate binding to TAM receptors. The sequence of the transcript variants of human Gas6 may be accessed at Genbank at NM_001143946.1. NM_001143945.1, and NM_000820.2, respectively.

The Gas6 binding portion provides isolated fragments thereof which specifically binds to a Gas6 protein. Isolated fragments of the Gas6 binding portion can bind to an epitope comprised in or presented by one or more amino acid regions that interact with Axl (eg., LRMFSGTPVIRLRFKRLQPT (SEQ ID NO: 90), EIVGRVTSSGP (SEQ ID NO: 91), RNLVIKVN (SEQ ID NO: 92), DAVMKIAVA (SEQ ID NO: 93), ERGLYHLNLTVGIPFH (SEQ ID NO: 94), and WLNGEDTTIQETVVNRM (SEQ ID NO: 95) or belong to Gas6 (L295-T317, E356-P372, R389-N396, D398-A406, E413-H429, and W450-M468). In addition, the isolated fragments of the Gas6 binding portion is capable of inhibiting or competing with the binding between TAM and Gas6. Of the TAM receptors, wild-type Axl or soluble Axl (sAxl) variants have the highest in vitro demonstrated affinity for Gas6 with K_{D} of 1.0 nM/L, followed by Tyro-3 with roughly equal affinity, and MerTK at least 10-fold lower of the K_{D} value.

The Gas6 binding portions can be any conformation which inhibit the Gas6/TAM pathway by neutralizing Gas6. The use of the Gas6 binding portion to neutralize the Gas6 can be a better strategy than blocking the TAM receptors with an antagonistic molecule because upregulation of Gas6 production induced by TAM antagonistic molecules in a negative feedback loop can be speculated. The Gas6 binding portions can be any smaller fragments of extracellular domain of TAM receptors; partially or fully humanized antibodies, or chimeric antibodies; monoclonal or polyclonal antibodies; fragments of the isolated antibodies of the Gas6 binding portion can contain a region of an antibody (either in the context of an antibody scaffold or a non-antibody scaffold) that is sufficient or necessary for a recognizable specific binding of the polypeptide towards Gas6, one or more CDRs of heavy chains or light chains or combinations thereof, a polypeptide containing a single chain antibody, variable regions only or variable regions in combination with part of Fc regions (eg. CH1 region), or minibodies (eg. VL-VH-CH3) or diabodies.

An Axl receptor represents a structure shared among TAM family members comprising an intracellular tyrosine kinase domain and an extracellular region that juxtaposes immunoglobulin (Ig) repeats and fibronectin type III (FnIII) repeats. The extracellular Ig and Fn motifs are believed to be important in cell adhesion and migration, indicating a mean through which the Axl oncogene contributes to tumor invasiveness and metastasis. Axl transduces signals from the extracellular matrix into the cytoplasm by binding growth factors such as vitamin K-dependent protein Gas6. This interaction activates Axl by causing dimerization and auto-phosphorylation.

A Gas6 binding portion in the fusion protein can include any smaller fragments of the extracellular domain of Axl that retain the ability to bind to a Gas6 which otherwise would bind to a TAM receptor. For example, an Axl fragment spanning the two N-terminal Ig domains (denoted Ig1 and Ig2) and lacking carbohydrate modifications retains full Gas6 binding activity. A suitable Gas6 binding portion for use in the fusion protein excludes at least the cytoplasmic domain of Axl, and preferably all or the majority of the transmembrane domain of Axl, and includes a portion of the extracellular domain of Axl, up to the entire extracellular domain. Preferably, the portion of the extracellular domain includes at least the major Gas6 binding surface of Axl, and in other embodiments, contains at least the Ig1 and Ig2 domains of Axl, or residues therein that form a conformational structure sufficient to bind to a Gas6.

The native sequence of Axl shown in SEQ ID NO: 1 comprises an Ig1, an Ig2, an FnIII, and an intracellular domain, where the Ig1 domain sequence is residues 27-128 (SEQ ID NO: 61), the Ig2 domain sequence is residues 139-222 (SEQ ID NO: 62), the FnIII domain sequence is residues 225-332 (SEQ ID NO: 63), and residues 333-427 (SEQ ID NO: 64), the intracellular domain sequence is residues 473-894 (SEQ ID NO: 65), the tyrosine residues at 779, 821, and 866 become autophosphorylated upon receptor dimerization and serve as docking sites for intracellular signaling molecules. The native cleavage site to release the sAxl lies between residues 437-451 (SEQ ID NO: 66).

The Gas6 binding portion of the fusion protein disclosed in this disclosure has been modified or rearranged based on the native sequence of Axl. For example, the Gas6 binding portion can include Ig1 and Ig2, and exclude FnIII, where the sequence is SEQ ID NO: 2.

The Gas6 binding portion of the fusion protein can include one or more amino acid modifications within the wild-type sAxl, for example, one or more amino acid modifications that increase its affinity for Gas6. Amino acid modifications include any naturally occurring or man-made amino acid modifications known or later discovered in the field. Amino acid modifications include any naturally occurring mutation, for example, substitution, deletion, addition, insertion, etc; replacing existing amino acid with another amino acid, for example, a conservative equivalent thereof; and replacing one or more existing amino acids with non-natural amino acids or inserting one or more non-natural amino acids. In some embodiments, amino acid modifications can include at least 1, 2, 3, 4, 5, or 6 or 10 amino acid mutations or changes. In some exemplary embodiments, one or more amino acid modifications can be used to alter properties of the sAxl, for example, affecting the stability, binding activity, specificity, and/or thermal stability etc. In some other embodiments, a sAxl lacks the transmembrane domain, and optionally the intracellular domain.

For the sufficient binding activity to Gas6, the modification normally lies between the extracellular and transmembrane domains, generally between residues 19-437 in SEQ ID NO: 1, but which may comprise or consist essentially of a truncated version from residue 19, 25, 30, 35, 40, 45, or 50 to residue 132, 321, 350, 375, 400, 410, 420, 430, 440, or 450. In some embodiments, the Gas6 binding portion includes one or more amino acid modifications within one or more regions of residue 18-130, 10-135, 15-45, 60-65, 70-80, 85-90, 91-99, 104-110, 111-120, 125-130, 21-132, 21-121, 26-132, or 26-121 of wild-type Axl (SEQ ID NO: 1). In other embodiments, the Gas6 binding portion includes one or more amino acid modifications within one or more regions of residue 20-130, 37-124, or 141-212 of wild-type Axl (SEQ ID NO: 1). In some other embodiments, the Gas6 binding portion includes one or more amino acid modifications at one or more modifications of residue 19, 23, 26, 27, 32, 33, 38, 44, 61, 65, 72, 74, 78, 79, 86, 87, 88, 90, 92, 97, 98, 105, 109, 112, 113, 116, 118, 127, or 129 of wild-type Axl (SEQ ID NO: 1), for example, A 19T, T23M, E26G, E27G/K. G32S, N33S, T38I, T44A, H61Y, D65N, A72V, S74N, Q78E, V79M, Q86R. D87G. D88N, I90M/V. V92A/G/D, 197R, T98A/P. T105M, Q109R, V112A, F113L, H116R. T118A, G127R/E, E129K and a combination thereof. In yet some other embodiments, the Gas6 binding portion includes one or more amino acid modifications at residue 32, 87, 92, or 127 of wild-type Axl (SEQ ID NO: 1) or a combination thereof, for example, G32S, D87G, V92A, and/or G127R. In still some other embodiments, the Gas6 binding portion includes one or more amino acid modifications at residue 26, 79, 92, 127 of wild-type Axl (SEQ ID NO: 1) or a combination thereof, for example, E26G, V79M, V92A, and/or G127E.

The Gas6 binding portion can be further modified, for example, joined to a wide variety of other oligopeptides or proteins for a variety of purposes. Various posttranslation or posttranscription modifications can be carried out with respect to the Gas6 binding portion of the fusion protein. Such modifications can include chemical derivatization of polypeptides, such as acetylation, amidation, carboxylation, etc. Such modifications can include modifications of glycosylation, for example, by exposing the polypeptide to mammalian glycosylating or deglycosylating enzymes. Such modifications can also include phosphorylating certain amino acid residues, for example, phosphotyrosine, phosphoserine, or phosphothreonine. Furthermore, by employing the appropriate coding sequences, one may provide farnesylation or prenylation. In some embodiments, the Gas6 binding portion can be PEGylated, where the polyethylaeneoxy group provides for enhanced half-life in the blood stream. The Gas6 binding portion can also be combined with other proteins, such as the Fc of an IgG isotype, which can be complement binding, with a toxin, such as ricin, abrin, diphtheria toxin, or the like, or with specific binding agents that allow targeting to specific moieties on a target cell. In some other embodiments, the Gas6 binding portion can be modified to improve their resistance to proteopytic degradation or to optimize solubility properties or to render them more suitable as a therapeutic agent. For example, the Gas6 binding portion can further include analogs of a sAxl variant containing residues other than naturally occurring L-amino acids, such as D-amino amino acids or non-naturally occurring synthetic amino acids. The D-amino acids may be substituted for some or all of the amino acid residues. In yet some other embodiments, the Gas6 binding portion can include two, three, four, five, or six same or different sAxl variants linked covalently or non-covalently, so that they will have the appropriate size but avoid unwanted aggregation.

In some embodiments, the Gas6 binding portion is a fusion protein, for example, fused in frame with a second polypeptide. The second polypeptide can be part or whole of Fc region, any suitable polypeptides that is substantially similar to Fc, or part or whole of an albumin protein in order to increase the size of the fusion protein for prolonging the half-life of the fusion protein. In other embodiments, the second polypeptide is useful for handling the Gas6 binding portion, for example, for purification of the Gas6 binding portion, or for stabilization of the Gas6 binding portion in vitro or in vivo. For example, the second polypeptide can be a marker sequence, such as a hexa-histidine peptide, to facilitate purification of the fused polypeptides. In addition, a region of additional amino acids, particularly charged amino acids, may be added to the N-terminus of the polypeptide to improve stability and persistence during purification from the host cell or subsequent handling and storage. Further, fusion proteins having disulfide-linked dimeric structures can also be more efficient in binding and neutralizing other molecules than the monomeric secreted protein or protein fragment alone.

### Antigen Binding Portion:

In some embodiments of the present disclosure, the antibody or antigen-binding fragment thereof comprises complementarity determining regions (CDRs) of a heavy chain variable region (VH) and complementarity determining regions of a light chain variable region (VL), wherein the complementarity determining regions of the heavy chain variable region comprise VH-CDR1, VH-CDR2 and VH-CDR3, and the complementarity determining regions of the light chain variable region comprise VL-CDR1, VL-CDR2 and VL-CDR3.

Examples of the antibody or antigen-binding fragment thereof include, but are not limited to an antibodies (a) to (k) or antigen-binding fragment thereof, 3G10 of U.S. Pat. No. 7,943,743, 12A4 of U.S. Pat. No. 7,943,743, 10A5 of U.S. Pat. No. 7,943,743, 5F8 of U.S. Pat. No. 7,943,743, 10H10 of U.S. Pat. No. 7,943,743, 1B12 of U.S. Pat. No. 7,943,743, 7H1 of U.S. Pat. No. 7,943,743, 11E6 of U.S. Pat. No. 7,943,743, 12B7 of U.S. Pat. No. 7,943,743, 13G4 of U.S. Pat. No. 7,943,743, MDX-1105, MEDI-4736, atezolizumab, durvalumab, avelumab, MDX-1105, envafolimab, cosibelimab, CK-301, CS-1001, SHR-1316, CBT-502, or BGB-A333.

In some embodiments of the disclosure, the antibody (a) or antigen-binding fragment thereof comprises VH-CDR1 of GYSITSDYWN (SEQ ID NO: 3) or a substantially similar sequence thereof; VH-CDR2 of YISYTGSTYYNPSLKS (SEQ ID NO: 4) or a substantially similar sequence thereof; VH-CDR3 of RGEWLSPFAY (SEQ ID NO: 5) or a substantially similar sequence thereof); VL-CDR1 of KSSQSLLYSSNQKNSLA (SEQ ID NO: 10) or a substantially similar sequence thereof; VL-CDR2 of WASTRES (SEQ ID NO: 11) or a substantially similar sequence thereof; and VL-CDR3 of QQYYTYPFT (SEQ ID NO: 12) or a substantially similar sequence thereof.

In some embodiments of the disclosure, the antibody (a) or antigen-binding fragment thereof comprises VH-CDR1 of GYSITSDYWD (SEQ ID NO: 96) or a substantially similar sequence thereof; VH-CDR2 of YISYTGSTYYNPSLRS (SEQ ID NO: 97) or a substantially similar sequence thereof; VH-CDR3 of RGGWLSPFVY (SEQ ID NO: 98) or a substantially similar sequence thereof); VL-CDR1 of KSRQSLLESSNQKNSLA (SEQ ID NO: 99) or a substantially similar sequence thereof; VL-CDR2 of WASTRES (SEQ ID NO: 11) or a substantially similar sequence thereof; and VL-CDR3 of QQYYTYPFT (SEQ ID NO: 12) or a substantially similar sequence thereof.

In some embodiments of the disclosure, the VH of the antibody (a) or antigen-binding fragment thereof comprises a framework represented by the formula: (HC-FR1)-(VH-CDR1)-(HC-FR2)-(VH-CDR2)-(HC-FR3)-(VH-CDR3)-(HC-FR4). In some embodiments of the disclosure, the HC-FR1 is EVQLQESGPGLVKPSQTLSLTCTVS (SEQ ID NO: 6) or a substantially similar sequence thereof; the HC-FR2 is WIRKPPGKGLEYMG (SEQ ID NO: 7) or a substantially similar sequence thereof; the HC-FR3 is RITISRDTSKNQYSLKLSSVTAADTAVYYCAR (SEQ ID NO: 8) or a substantially similar sequence thereof; and the HC-FR4 is WGQGTLVTVSS (SEQ ID NO: 9) or a substantially similar sequence thereof.

In some embodiments of the disclosure, the VL of the antibody (a) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4). In some embodiments of the disclosure, the LC-FR1 is DIQMTQSPSSLS ASVGDRVTITC (SEQ ID NO: 13) or a substantially similar sequence thereof; the LC-FR2 is WYQQKPGKAPKLLIY (SEQ ID NO: 14) or a substantially similar sequence thereof; the LC-FR3 is GVPSRFSGSGSGTDFTLTISSLQ PEDFATYYC (SEQ ID NO: 15) or a substantially similar sequence thereof; and the LC-FR4 is FGQGTKLEIK (SEQ ID NO: 16) or a substantially similar sequence thereof.

In some embodiments of the disclosure, the VL of the antibody (a) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4). In some embodiments of the disclosure, the LC-FR1 is DIVMTQSPDSLAVSLGERATINC (SEQ ID NO: 100) or a substantially similar sequence thereof; the LC-FR2 is WYQQKPGQPPKLLIY (SEQ ID NO: 101) or a substantially similar sequence thereof; the LC-FR3 is GVPDRFSGSGSGTDFTLTISSLQAEDVAVYYC (SEQ ID NO: 102) or a substantially similar sequence thereof; and the LC-FR4 is FGQGTKLEIK (SEQ ID NO: 16) or a substantially similar sequence thereof.

In some embodiments of the disclosure, the antibody (a) or antigen-binding fragment disclosed above is the selected humanization clone of its corresponding mouse clone, wherein the mouse clone comprises complementarity determining regions of a heavy chain variable region, complementarity determining regions of a light chain variable region, and frameworks represented by the formulas: (HC-FR1)-(VH-CDR1)-(HC-FR2)-(VH-CDR2)-(HC-FR3)-(VH-CDR3)-(HC-FR4) and (LC-FR1)-(VL-CDR1)-(LC-FR2)-(VL-CDR2)-(LC-FR3)-(VL-CDR3)-(LC-FR4). The said mouse clone comprises VH-CDR1 of GYSITSDYWN (SEQ ID NO: 3) or a substantially similar sequence thereof; VH-CDR2 of YISYTGSTYYNPSLKS (SEQ ID NO: 4) or a substantially similar sequence thereof; VH-CDR3 of RGEWLSPFAY (SEQ ID NO: 5) or a substantially similar sequence thereof); VL-CDR1 of KSSQSLLYSSNQKNSLA (SEQ ID NO: 10) or a substantially similar sequence thereof; VL-CDR2 of WASTRES (SEQ ID NO: 11) or a substantially similar sequence thereof; and VL-CDR3 of QQYYTYPFT (SEQ ID NO: 12) or a substantially similar sequence thereof; the HC-FR1 is QVQLQESGPGLAKPSQTLSLTCSVT (SEQ ID NO: 103) or a substantially similar sequence thereof; the HC-FR2 is WIRKFPGNKLEFMG (SEQ ID NO: 104) or a substantially similar sequence thereof; the HC-FR3 is RISITRDTSKNQYYLQLNSVTTEDTATYYCAR (SEQ ID NO: 105) or a substantially similar sequence thereof; and the HC-FR4 is WGQGTLVTVSA (SEQ ID NO: 106) or a substantially similar sequence thereof; the LC-FR1 is DIVMSQSPSSLGVSVGEKITMSC (SEQ ID NO: 107) or a substantially similar sequence thereof; the LC-FR2 is WYQQKPGQSPKLLIY (SEQ ID NO: 108) or a similar substantially sequence thereof; the LC-FR3 is GVPDRFTGSGSGTDFTLTISSVKSEDLAVYYC (SEQ ID NO: 109) or a substantially similar sequence thereof; and the LC-FR4 is FGAGTNLELK (SEQ ID NO: 110) or a substantially similar sequence thereof.

In some embodiments of the disclosure, the antibody (a) or antigen-binding fragment disclosed above is the selected humanization clone of its corresponding mouse clone, wherein the mouse clone comprises complementarity determining regions of a heavy chain variable region, complementarity determining regions of a light chain variable region, and frameworks represented by the formulas: (HC-FR1)-(VH-CDR1)-(HC-FR2)-(VH-CDR2)-(HC-FR3)-(VH-CDR3)-(HC-FR4) and (LC-FR1)-(VL-CDR1)-(LC-FR2)-(VL-CDR2)-(LC-FR3)-(VL-CDR3)-(LC-FR4). The said mouse clone comprises VH-CDR1 of GYSITSDYWD (SEQ ID NO: 96) or a substantially similar sequence thereof; VH-CDR2 of YISYTGSTYYNPSLRS (SEQ ID NO: 97) or a substantially similar sequence thereof; VH-CDR3 of RGGWLSPFVY (SEQ ID NO: 98) or a substantially similar sequence thereof); VL-CDR1 of KSRQSLLFSSNQKNSLA (SEQ ID NO: 99) or a substantially similar sequence thereof; VL-CDR2 of WASTRES (SEQ ID NO: 11) or a substantially similar sequence thereof; and VL-CDR3 of QQYYTYPFT (SEQ ID NO: 12) or a substantially similar sequence thereof; the HC-FR1 is EVQLQESGPGLTKPSQTLSLTCSVT (SEQ ID NO: 111) or a substantially similar sequence thereof; the HC-FR2 is WIRKFPGNKLEYMG (SEQ ID NO: 112) or a substantially similar sequence thereof; the HC-FR3 is RISITRDTSKNQYYLQLSSVTSEDSATYYCAR (SEQ ID NO: 113) or a substantially similar sequence thereof; and the HC-FR4 is WGQGTLVTVSA (SEQ ID NO: 114) or a substantially similar sequence thereof; the LC-FR1 is DTVMSQSPSSLGVSVGERVTLTC (SEQ ID NO: 115) or a substantially similar sequence thereof; the LC-FR2 is WYQQKPGQSPKLLIY (SEQ ID NO: 116) or a substantially similar sequence thereof; the LC-FR3 is GVPDRFTGSGSGTDFTLTISSVKSEDLAVYYC (SEQ ID NO: 117) or a substantially similar sequence thereof; and the LC-FR4 is FGAGTSLELK (SEQ ID NO: 118) or a substantially similar sequence thereof.

In some embodiments of the disclosure, the antibody (b) or antigen-binding fragment thereof comprising VH-CDR1 of SYIMM (SEQ ID NO: 17) or a substantially similar sequence thereof; VH-CDR2 of SIYPSGGITFYADTVKG (SEQ ID NO: 18) or a substantially similar sequence thereof; VH-CDR3 of IKLGTVTTVDY (SEQ ID NO: 19) or a substantially similar sequence thereof. VL-CDR1 of TGTSSDVGGYNYVS (SEQ ID NO: 20) or a substantially similar sequence thereof; VL-CDR2 of DVSNRPS (SEQ ID NO: 21) or a substantially similar sequence thereof; and VL-CDR3 of SSYTSSSTRV (SEQ ID NO: 22) or a substantially similar sequence thereof.

In some embodiments of the disclosure, the antibody (c) or antigen-binding fragment thereof comprising VH-CDR1 of MYMMM (SEQ ID NO: 23) or a substantially similar sequence thereof; VH-CDR2 of SIYPSGGITFYADSVKG (SEQ ID NO: 24) or a substantially similar sequence thereof; VH-CDR3 of IKLGTVTTVDY (SEQ ID NO: 25) or a substantially similar sequence thereof. VL-CDR1 of TGTSSDVGAYNYVS (SEQ ID NO: 26) or a substantially similar sequence thereof; VL-CDR2 of DVSNRPS (SEQ ID NO: 27) or a substantially similar sequence thereof; and VL-CDR3 of SSYTSSSTRV (SEQ ID NO: 28) or a substantially similar sequence thereof.

In some embodiments of the disclosure, the antibody (d) or antigen-binding fragment thereof comprising VH-CDR1 of SYIMM (SEQ ID NO: 29) or a substantially similar sequence thereof; VH-CDR2 of SIYPSGGITFYAPTVKG (SEQ ID NO: 30) or a substantially similar sequence thereof; VH-CDR3 of IKLGTVTTVDY (SEQ ID NO: 31) or a substantially similar sequence thereof. VL-CDR1 of TGTSSDVGGYNYVS (SEQ ID NO: 32) or a substantially similar sequence thereof; VL-CDR2 of DVSNRPS (SEQ ID NO: 33) or a substantially similar sequence thereof; and VL-CDR3 of SSYTSSSTRV (SEQ ID NO: 34) or a substantially similar sequence thereof.

In some embodiments of the disclosure, the VH of the antibody (d) or antigen-binding fragment thereof comprises a framework represented by the formula: (HC-FR1)-(VH-CDR1)-(HC-FR2)-(VH-CDR2)-(HC-FR3)-(VH-CDR3)-(HC-FR4). In some embodiments of the disclosure, the HC-FR1 is EVQLLESGGGLVQPGGSLRLSCAASGFTGS (SEQ ID NO: 35) or a substantially similar sequence thereof; the HC-FR2 is WVRQAPGKGLEWVS (SEQ ID NO: 36) or a substantially similar sequence thereof; the HC-FR3 is RFTISRDNSKNTLYLQMNSLRAEDTAVYYCAR (SEQ ID NO: 37) or a substantially similar sequence thereof; and the HC-FR4 is WGQGTLVTVSS (SEQ ID NO: 38) or a substantially similar sequence thereof. In some embodiments of the disclosure, the VL of the antibody (d) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4). In some embodiments of the disclosure, the LC-FR1 is QSALTQPASVSGSPGQSITISC (SEQ ID NO: 39) or a substantially similar sequence thereof; the LC-FR2 is WYQQHPGKAPKLMIY (SEQ ID NO: 40) or a substantially similar sequence thereof; the LC-FR3 is GVSNRFSGSKSGNTASLTISGLQAEDEADYYC (SEQ ID NO: 41) or a substantially similar sequence thereof; and the LC-FR4 is FGTGTKVTVL (SEQ ID NO: 42) or a substantially similar sequence thereof.

In some embodiments of the disclosure, the antibody (c) or antigen-binding fragment thereof comprising VH-CDR1 of SYIMM (SEQ ID NO: 43) or a substantially similar sequence thereof; VH-CDR2 of SIYPSGGITGYADTVKG (SEQ ID NO: 44) or a substantially similar sequence thereof; VH-CDR3 of IKLGTVTTVDY (SEQ ID NO: 45) or a substantially similar sequence thereof, VL-CDR1 of TGTSSDVGGYNYVS (SEQ ID NO: 46) or a substantially similar sequence thereof; VL-CDR2 of DVSNRPS (SEQ ID NO: 47) or a substantially similar sequence thereof; and VL-CDR3 of SSYTSSSTRV (SEQ ID NO: 48) or a substantially similar sequence thereof.

In some embodiments of the disclosure, the antibody (f) or antigen-binding fragment thereof comprising a heavy chain of EVQLLESGGGLVQPGGSLRLSCAASGFTFSSYIMMVWRQAPGKGLEWVSSIYPSGGITFY ADWKGRFTISRDNSKNTLYLQMNSLRAEDTAVYYCARIKLGTVTTVDYWGQGTLVYVS S (SEQ ID NO: 49) or a substantially similar sequence thereof and a light chain of QSALTQPASVSGSPGQSITISCTGTSSDVGGYNYVSWYQQHPGKAPKLMIYDVSNRPSGV SNRFSGSKSGNTASLTISGLOAEDEADYYCSSYTSSSTRVFGTGTKVTVL (SEQ ID NO: 50) or a substantially similar sequence thereof.

In some embodiments of the disclosure, the antibody (g) or antigen-binding fragment thereof comprising a heavy chain of EVQLLESGGGLVQPGGSLRLSCAASGFTFSMYMMMWVRQAPGKGLEVWSSIYPSGGITF YADSVKGRFTISRDNSKNTLYLQMNSLRAEDTAIYYCARIKLGTVTTVDYWGQGTLVTV SS (SEQ ID NO: 51) or a substantially similar sequence thereof and a light chain of QSALTQPASVSPGQSITISCTGTSSDVGAYNYVSWYQQHPGKAPKLMIYDVSNRPSGVSN RFSGSKSGNTASLTISGLQAEDEADYYCSSYTSSSTRVFGTGTKVTVL (SEQ ID NO: 52) or a substantially similar sequence thereof.

In some embodiments of the disclosure, the antibody (h) or antigen-binding fragment thereof comprising heavy a chain of EVOLVESGGGLVQPGGSLRLSCAASGFTFSDSWIHWVRQAPGKGLEWVAWISPYGGSTY YADSVKGRFTISADTSKNTAYLQMNSLRAEDTAVYYCARRHWPGGFDYWGQGTLVTVS S (SEQ ID NO: 53) or a substantially similar sequence thereof and a light chain of DIQMTQSPSSLSASVGDRVTITCRASQDVSTAVAWYQQKPGKAPKLLIYSASFLYSGVPS RFSGSGSGTDFTLTISSLQPEDFATYYCQQYLYHPATFGQGTKVEIKR (SEQ ID NO: 54) or a substantially similar sequence thereof.

In some embodiments of the disclosure, the antibody (i) or antigen-binding fragment thereof comprising a heavy chain of EVOLVESGGGLVQPGGSLRLSCAASGFTFSDSWIHWVRQAPGKGLEWVAWISPYGGSTY YADSVKGRFTISADTSKNTAYLQMNSLRAEDTAVYYCARRHWPGGFDYWGQGTLVTVS S (SEQ ID NO: 55) or a substantially similar sequence thereof and a light chain of DIQMTQSPSSLSASVGDRVTITCRASQDVSTAVAWYQQKPGKAPKLLIYSASFLYSGVPS RFSGSGSGTDFTLTISSLQPEDFATYYCQQYLYHPATFGQGTKVEIKR (SEQ ID NO: 56) or a substantially similar sequence thereof.

In some embodiments of the disclosure, the antibody (j) or antigen-binding fragment thereof comprising a chain heavy of EVOLVESGGGLVQPGGSLRLSCAASGFTFSDSWIHWVRQAPGKGLEWVAWISPYGGSTY YADSVKGRFTISADTSKNTAYLQMNSLRAEDTAVYYCARRHWPGGFDYWGQGTLVTVS A (SEQ ID NO: 57) or a substantially similar sequence thereof and a light chain of DIQMTQSPSSLSASVGDRVTITCRASQDVSTAVAWYQQKPGKAPKLLIYSASFLYSGVPS RFSGSGSGTDFTLTISSLQPEDFATYYCQQYLYHPATFGQGTKVEIKR (SEQ ID NO: 58) or a substantially similar sequence thereof.

In some embodiments of the disclosure, the antibody (k) or antigen-binding fragment thereof comprising a heavy chain encoded by SEQ ID NO: 59; and a light chain encoded by SEQ ID NO: 60.

Another embodiment, the antibody binds to human, mouse, or cynomolgus monkey PD-L1. In a specific disclosure, the antibody is capable of blocking the interaction between human, mouse, or cynomolgus monkey PD-L1 and the respective human, mouse, or cynomolgus monkey PD-1 receptors.

Another embodiment, the antibody binds to human PD-L1 with a KD of 5×10⁹ M or less, preferably with a KD of 2×10⁹ M or less, and even more preferred with a KD of 1×10⁹ M or less.

Yet other embodiment relates to an anti-PD-L1 antibody or antigen-binding fragment thereof which binds to a functional epitope including residues Y56 and D61 of human PD-L1.

In specific embodiment, the antibody binds to a conformational epitope, including residues 54-66 and 112-122 of human PD-L1.

In yet other embodiments, the framework sequences are derived from human consensus framework sequences or human germline framework sequences.

In still further embodiments, the light chain framework sequences are lambda light chain sequences.

### Constant Region:

In some embodiments of the present disclosure, the antibody or antigen-binding fragment thereof comprises a heavy chain constant region and a light chain constant region.

In still further embodiments, the heavy chain variable region polypeptide, antibody, or antibody fragment further includes at least a CH1 domain. In more specific embodiments, the heavy chain variable region polypeptide, antibody, or antibody fragment further includes a CH1, CH2, and a CH3 domain.

In still further embodiments, the variable region light chain, antibody, or antibody fragment further includes a CL domain.

In still further embodiments, the antibody further includes a CH1, a CH2, a CH3, and a CL domain.

In some embodiments of the disclosure, the proteins and peptides of the antigen binding portion include a constant region of an immunoglobulin or a fragment, analog, variant, mutant, or derivative of the constant region. In preferred embodiment herein, the constant region is derived from a human immunoglobulin heavy chain, for example, IgG1, IgG2, IgG3, IgG4, or other classes. In some embodiment, the constant region includes a CH2 domain. In other embodiments, the constant region includes CH2 and CH3 domains or includes hinge-CH2-CH3. Alternatively, the constant region can include all or a portion of the hinge region, the CH2 domain and/or the CH3 domain.

In some embodiment, the constant region contains a mutation that reduces affinity for an Fc receptor or reduces Fc effector function. For example, the constant region can contain a mutation that eliminates the glycosylation site within the constant region of an IgG heavy chain. In some embodiment herein, the constant region contains mutations, deletions, or insertions at an amino acid position corresponding to L234, L235, G236, G237, N297, or P331 of IgG1. In particular embodiment, the constant region contains a mutation at an amino acid position corresponding to N297 of IgG1. In alternative embodiment herein, the constant region contains mutations, deletions, or insertions at an amino acid position corresponding to L281, L282, G283, G284, N344, or P378 of IgG1.

In some embodiment herein, the constant region contains a CH2 domain derived from a human IgG2 or IgG4 heavy chain. Preferably, the CH2 domain contains a mutation that eliminates the glycosylation site within the CH2 domain. In some embodiment, the mutation alters the N within the QFNS amino acid sequence within the CH2 domain of the IgG2 or IgG4 heavy chain. Preferably, the mutation changes the N to a G. Alternatively, the mutation alters both the F and the N within the QFNS amino acid sequence. In some embodiment, the QFNS amino acid sequence is replaced with a QAQS amino acid sequence. The N within the QFNS amino acid sequence corresponds to N297 of IgG1.

In other embodiment, the constant region includes a CH2 domain and at least a portion of a hinge region. The hinge region can be derived from an immunoglobulin heavy chain, for example, IgG1, IgG2, IgG3, IgG4, or other classes. Preferably, the hinge region is derived from human IgG1. IgG2, IgG3, IgG4, or other suitable classes. More preferably, the hinge region is derived from a human IgG1 heavy chain. In some embodiment the C in the PKSCDK amino acid sequence of the IgG1 hinge region is altered. In preferred embodiment, the PKSCDK amino acid sequence is replaced with a PKSSDK amino acid sequence. In some embodiment, the constant region includes a CH2 domain derived from a first antibody isotype and a hinge region derived from a second antibody isotype. In specific embodiment, the CH2 domain is derived from a human IgG2 or IgG4 heavy chain, while the hinge region is derived form an altered human IgG1 heavy chain.

The alteration of amino acids near the junction of the Fe portion and the non-Fc portion can dramatically increase the serum half-life of the Fc fusion protein (PCT publication WO 01/58957). Accordingly, the junction region of a protein or polypeptide of the present disclosure can contain alterations that, relative to the naturally-occurring sequences of an immunoglobulin heavy chain and erythropoietin, preferably lie within about 10 amino acids of the junction point. These amino acid changes can cause an increase in hydrophobicity. In some embodiment, the constant region is derived from an IgG sequence in which the C-terminal K residue is replaced. Preferably, the C-terminal K of an IgG sequence is replaced with a non-K amino acid, such as A or L, to further increase serum half-life. In other embodiment, the constant region is derived from an IgG sequence in which the LSLS amino acid sequence near the C-terminal of the constant region is altered to eliminate potential junctional T cell epitopes. For example, in some embodiment, the LSLS amino acid sequence is replaced with an ATAT amino acid sequence. In other embodiment herein, the amino acids within the LSLS segment are replaced with other amino acids such as G or P. Detailed methods of generating amino acid substitutions of the LSLS segment near the C-terminal of an IgG1, IgG2, IgG3, IgG4, or other classes have been described in US patent publication 2003/0166877.

Suitable hinge regions for the present disclosure can be derived from IgG1, IgG2, IgG3, IgG4, and other classes. The IgG1 hinge region has three C, two of which are involved in disulfide bonds between the two heavy chains of the antibody. These same Cs permit efficient and consistent disulfide bonding formation between Fc portions. In some embodiment herein, the first C within the human IgG1 hinge region is mutated to another amino acid, preferably S. The IgG2 isotype hinge region has four disulfide bonds that tend to promote oligomerization and possibly incorrect disulfide bonding during secretion in recombinant systems. A suitable hinge region can be derived from an IgG2 hinge; the first two Cs are each preferably mutated to another amino acid. The hinge region of IgG4 is known to form interchain disulfide bonds inefficiently. However, a suitable hinge region for the fusion protein can be derived from the IgG4 hinge region, preferably containing a mutation that enhances correct formation of disulfide bonds between heavy chain-derived moieties.

In accordance with the current disclosure, the constant region can contain CH2 and/or CH3 domains and a hinge region that are derived from different antibody isotypes, for example, a hybrid constant region. For example, in some embodiment, the constant region contains CH2 and/or CH3 domains derived from IgG2 or IgG4 and a mutant hinge region derived from IgG1. Alternatively, a mutant hinge region from another IgG subclass is used in a hybrid constant region. For example, a mutant form of the IgG4 hinge that allows efficient disulfide bonding between the two heavy chains can be used. A mutant hinge can also be derived from an IgG2 hinge in which the first two Cs are each mutated to another amino acid. Assembly of such hybrid constant regions has been described in US patent publication NO. 2003/0044423.

In accordance with the current disclosure, the constant region can contain one or more mutations described herein. The combinations of mutations in the Fc portion can have additive or synergistic effects on the prolonged serum half-life and increased in vivo potency of the fusion protein. Thus, in exemplary disclosure, the constant region can contain (i) a region derived from an IgG sequence in which the LSLS amino acid sequence is replaced with an ATAT amino acid sequence; (ii) a C-terminal K residue is replaced with an A; (iii) a CH2 domain and a hinge region that are derived from different antibody isotypes, for example, a CH2 domain can be derived from an IgG2, while a hinge region an IgG1; and (iv) a mutation that eliminates the glycosylation site within the IgG2-derived CH2 domain, for example, a QAQS instead of the QFNS amino acid sequence within the IgG2-derived CH2 domain.

### Antigen-Binding Fragments:

In some embodiments of the present disclosure, the antigen-binding fragment comprises an Fab fragment, an F(ab′)₂ fragment, an ScFv fragment, a chimeric antibody, or a nanobody.

The proteins and polypeptides can also include antigen-binding fragments. Exemplary antibody fragments include scFv, Fv, Fab, F(ab)₂, and single domain VHH fragments such as those of camelid origin.

Single-chain antibody fragments, also known as single-chain antibodies (scFvs), are recombinant polypeptides which typically bind antigens or receptors; these fragments contain at least one fragment of a VH tethered to at least one fragment of a VL with or without one or more interconnecting linkers. Such a linker may be a short, flexible peptide selected to assure that the proper three-dimensional folding of the VH and VL domains occurs once they are linked so as to maintain the target molecule binding-specificity of the whole antibody from which the scFv is derived. Generally, the C-terminal of the VH or VL sequence is covalently linked by such a peptide linker to the amino acid terminal of a complementary VH and VL sequence.

Single-chain antibody fragments contain amino acid sequences having at least one of the CDRs of the whole antibodies described in this disclosure, but are lacking some or all of the constant domains of those antibodies. These constant domains are not necessary for antigen binding, but constitute a major portion of the structure of whole antibodies. Single-chain antibody fragments may therefore overcome some of the problems associated with the use of antibodies containing part or all of a constant domain. For example, single-chain antibody fragments tend to be free of undesired interactions between biological molecules and the heavy chain constant region, or other unwanted biological activity. In addition, single-chain antibody fragments are considerably smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely than whole antibodies to provoke an unwanted immune response in a recipient.

Fragments of antibodies that have the same or comparable binding characteristics to those of the whole antibody may also be present. Such fragments may contain one or both Fab fragments or the F(ab)₂ fragment. The antibody fragments may contain all six CDRs of the whole antibody, although fragments containing fewer than all of such regions, such as three, four, or five CDRs, are also functional.

Revive Immunosurveillance with the Anti-PD-L1 Antibody

T cell inhibition checkpoint receptors such as CTLA-4, PD-1, BTLA, LAG-3, TIM-3, and LAIR1 are highly expressed in inducible regulatory T cells and exhausted T cells. Therefore, the counterparts of the T cell inhibition checkpoint receptors such as PD-L1 (B7-H1), B7-DC, HVEM, TIM-4, B7-H3, and B7-H4 are found responsible for immune evasion in cancer.

The antibody moiety of the fusion protein helps direct the fusion protein to tumor and its microenvironment where the antibody moiety lifts the inhibition made by T cell inhibition checkpoints. To this end the present inventors have examined the anti-tumor efficacy of combining the Gas6 binding portion with both commercially available and in-house antibodies targeting PD-L1. The present inventors found that combining a Gas6 binding portion with an anti-PD-L1 antibody in one entity sacrificed the binding affinity and blocking functions in neither moiety of the anti-PD-L1×Gas6 binding portion. In addition, the anti-PD-L1×Gas6 binding portion exhibited remarkable anti-tumor activity beyond what was observed with a conventional combination strategy where the Gas6 binding portion and the anti-PD-L1 antibody were administered separately.

### Spatial Conformations:

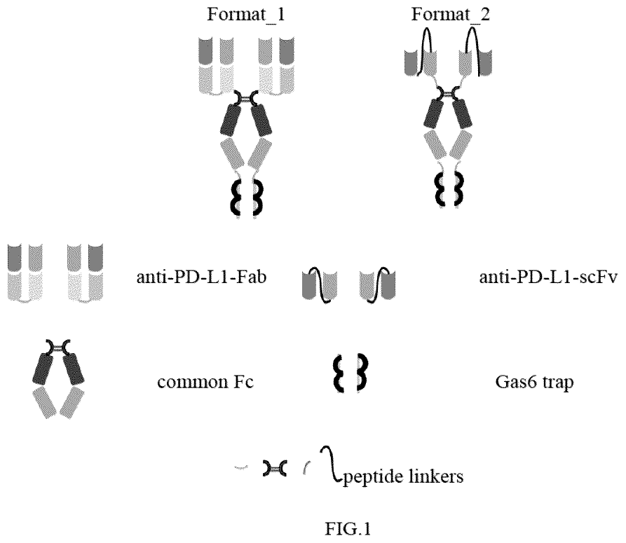

Either the light chain or the heavy chain of the anti-PD-L1 antibody or PD-L1-binding fragment thereof containing a Fc fragment can be fused to the Gas6 binding portion. The Gas6 binding portion thereof can be fused to either the N-terminus or the C-terminus of a chain of the anti-PD-L1 unit. The anti-PD-L1 unit thereof can either have a light chain and a separate heavy chain, or have a light chain and a heavy chain on a single protein chain (e.g., scFv). N-terminal fusion construction exhibited relatively lower productivity and significantly reduced biological activity compared to C-terminal fusion construction. The spatial conformations that have favorable yield and biological function in this disclosure based on the method of trial and error are presented in FIG. 1, wherein the Gas6 binding portion is fused to the heavy chain C-terminus of the anti-PD-L1 unit comprising either a light chain and a separate heavy chain (Format_1), or a light chain and a heavy chain on a single protein chain (Format_2); both with the N-terminus of Gas6 binding portion fused to the heavy chain C-terminus of anti-PD-L1 unit through peptide linkers.

### Peptide Linkers:

The disclosure comprises an Axl RTK extracellular domain fused to the C-terminus of the heavy chain of the anti-PD-L1 antibody, scFv or fragment thereof through a peptide linker. With reference to the ectodomain, the peptide linker which may be entirely an artificial linker, or include part of extracellular fragment N-terminal to the ectodomain IPPHVQKSVNNDMIVTDNNGAVKFP (SEQ ID NO: 67), should have a minimum length. If the distance is too short, the fusion protein has reduced stability or activity. The minimum length, in some embodiment, is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 amino acid residues. In some embodiments, the linker is not longer than 35, 40, 45, 50, 55, 60, 65, 70, 75, 80, 85, 90, 95, 100, 110, 120, 130, 140, 150, 170, or 200 amino acid residues.

Inclusion of a flexible linker, for instance, one or more GGGGS (SEQ ID NO: 68) units and, in some embodiments, can be useful for the stability and/or activity of the BfAb. In some embodiments, the flexible linker includes at least 40%, 50%, 60%, 70%, or 80% glycine. In some embodiments, the flexible linker includes one or more serine. In some embodiments, the flexible linker includes 1, 2, 3, 4, 5, or 6 of SEQ ID NO: 68 repeats.

It is shown that, in some embodiments, the natural N-terminal fragment (SEQ ID NO: 67) can be replaced with a substitute peptide to increase stability, without sacrifice or even with improvement of activity. In some embodiments, the substitute peptide is different from SEQ ID NO: 67 but has at least 30%, 40%, 50%, 60%, 70%, 80%, or 90% sequence identity to SEQ ID NO: 67.

An example substitute peptide is IPPHVQXXVNNDMIVTDNXGAVKFP (SEQ ID NO: 69), wherein X is any amino acid except K, S, or N. In some embodiments, substitutions can be made to remove the rigid di-peptide PP, removal of potential cleavage sites QK, N, and/or K, include multiple glycine residues to increase flexibility, and/or reduce hydrophobic residues. One such example is TAGHTQTSTGGGAITTGTSGAGHGP (SEQ ID NO: 70) or a variant having at least 50%, 55%, 60%, 65%, 70%, 75%, 80%, 85%, 90%, or 95% sequence identity to SEQ ID NO: 70. In some embodiments, the variant includes at least 4 G, no PP di-peptide, no more than 3 hydrophobic amino acid residues selected from the group consisting of I, L, M, F, V, W, Y, and P. In some embodiments, the variant includes at least 5 G, and no more than 1 hydrophobic amino acid residue selected from the group consisting of I, L, M, F, V, W, Y, and P.

In some embodiments, the peptide linker includes a substitute peptide of SEQ ID NO: 67. In some embodiments, the peptide linker includes both a flexible linker and a substitute peptide. In some embodiments, the flexible linker is N-terminal to the substitute peptide. In some embodiments, the flexible linker is C-terminal to the substitute peptide.

In some embodiments, the fusion protein at least does not include the entire sequence of EEYNTSNPD (SEQ ID NO: 71). The fusion protein may have the entire SEQ ID NO: 71 removed from the extracellular domain of Gas6 binding portion. In some embodiments, the fusion protein does not include more than 1, 2, 3, 4, 5, 6, 7, or 8 amino acid residues of SEQ ID NO: 71.

### Methods:

The fusion protein can be produced by any suitable means known or later discovered in the field, for example, produced from eukaryotic or prokaryotic cells, synthesized in vitro, etc. Where the protein is produced by prokaryotic cells, it may be further processed by unfolding, for example, heat denaturation, DTT reduction, etc., and may be further refolded, using methods known in the art.

Methods of making antibodies are well known in the art and described herein. An antibody suitable for use in the fusion protein may be obtained from natural sources or produced by hybridoma, recombinant or chemical synthetic methods, including modification of constant region function by genetic engineering techniques. The antibody of the fusion protein may be of any isotype. In certain embodiments, both the variable and constant regions of the antigen binding portion of the fusion protein are fully human antibodies made using techniques as described herein, for example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140.

The polypeptides may be prepared by in vitro synthesis, using conventional methods including molecular cloning, antibody phage display library, or similar techniques. Various commercial synthetic apparatuses are available. By using synthesizers, naturally occurring amino acids may be substituted with unnatural amino acids. The particular sequence and the manner of preparation will be determined by convenience, economics, purity required, and the like.

The polypeptides may also be isolated and purified according to conventional methods of recombinant proteins. A lysate may be prepared from the expression host and purified using HPLC, exclusion chromatography, gel electrophoresis, affinity chromatography, or other purification technique.

Methods which are well known to those skilled in the art can be used to construct expression vectors containing coding sequences and appropriate transcriptional/translational control signals. These methods include, for example, in vitro recombinant DNA techniques, synthetic techniques, and in vivo recombination/genetic recombination. Alternatively, RNA capable of encoding the polypeptides of interest may be chemically synthesized. Direct chemical synthesis methods include, for example, phosphotriester methods, diethylphosphoramidite method, and the solid support method. Chemical synthesis produces a single stranded oligonucleotide. This can be converted to double stranded DNA by hybridization with a complementary sequence, or by polymerization with a DNA polymerase using the single strand as a template. While chemical synthesis of DNA is often limited to sequences of about 100 bases, longer sequences can be obtained by the ligation of shorter sequences. Alternatively, subsequences may be cloned and the appropriate subsequences cleaved using appropriate restriction enzymes.

The nucleic acids may be isolated and obtained in substantial purity. The nucleic acids of the disclosure can be provided as a linear molecule or within a circular molecule, and can be provided within autonomously replicating molecules or within molecules without replication sequences. Expression of the nucleic acids can be regulated by their own or by other regulatory sequences known in the art. The nucleic acids of the fusion protein can be introduced into suitable host cells using a variety of techniques available in the art, such as transferrin polycation-mediated DNA transfer, transfection with naked or encapsulated nucleic acids, liposome-mediated DNA transfer, intracellular transportation of DNA-coated latex beads, protoplast fusion, viral infection, electroporation, gene gun, calcium phosphate-mediated transfection, and the like.

### Therapeutic Indications:

The present disclosure further provides a method for treating, prophylactic treating and/or preventing a cancer in a subject in need thereof, comprising administering the subject with an effective amount of the fusion protein as disclosed herein.

The present disclosure further provides a method for neutralizing Gas6 in a sample, comprising contacting the sample with the fusion protein as disclosed herein.

As described herein, the antibodies, variants, or derivatives of the anti-PD-L1×Gas6 binding portion may be used in certain treatment and diagnostic methods.

The fusion protein is further directed to multifunctional molecule- or antibody-based therapies which involve administering the multifunctional molecules or the antibodies of the disclosure to a patient such as an animal, a mammal, and a human for treating one or more of the disorders or conditions described herein. Therapeutic compounds of the disclosure include, but not limited to, antibodies of the disclosure (including variants and derivatives thereof as described herein), and nucleic acids or polynucleotides encoding antibodies of the disclosure (including variants and derivatives thereof as described herein).

The fusion protein is a method for treating a cancer in a patient in need thereof. The method, in one embodiment, entails administering to the patient an effective amount of a fusion protein of the present disclosure. In some embodiments, at least one of the cancer cells or surrounding cells in the tumor microenvironment in the patient expresses, overexpresses, or is induced to express PD-L1 and/or Gas6 or Axl. Induction of PD-L1, Gas6, or Axl expression, for instance, can be done by administration of a tumor vaccine or radiotherapy.

Tumors that express the PD-L1 protein include those of bladder cancer, non-small cell lung cancer, renal cancer, breast cancer, urethral cancer, colorectal cancer, head and neck cancer, squamous cell cancer, Merkel cell carcinoma, gastrointestinal cancer, stomack cancer, oesophageal cancer, ovarian cancer, and small cell lung cancer. Tumors that benefit from Gas6/Axl axis include those of lung cancer, myeloid leukemia, uterine cancer, ovarian cancer, gliomas, melanoma, prostate cancer, breast cancer, gastric cancer, colon cancer, osterosarcoma, renal cell carcinoma, and thyroid cancer. In addition, because the Gas6 binding portion of the present disclosure acts as a ligand trap for Gas6, the composition and method of the disclosure are useful for the treatment of any cancer in which Axl is expressed, and potentially any cancer in which MerTK and/or Tyro-3 are expressed.

Accordingly, the present disclosed anti-PD-L1×Gas6 binding portion can be used for treating any one or more such cancers. Additional diseases or conditions associated with increased cell survival, that may be treated, prevented, diagnosed and/or prognosed with the fusion proteins or their variants or derivatives thereof include, but not limited to, progression, and/or metastases of malignancies and related disorders such as leukemia, polycythemia vera, lymphomas, multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, pancreatic cancer, thyroid cancer, endometrial cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, hepatoma, bile duct carcinoma, choriocarcinoma, seminoma, embryonal carcinoma, Wilm's tumor, cervical cancer, testicular tumor, bladder carcinoma, epithelial carcinoma, astrocytoma, medulloblastoma, craniopharyngioma, ependymoma, pinealoma, hemangioblastoma, acoustic neuroma, oligodendroglioma, menangioma, neuroblastoma, and retinoblastoma.

### Pharmaceutical Composition:

The present disclosure further provides a pharmaceutical composition, including: an effective amount of the fusion protein as disclosed herein or the genetically engineered cell as disclosed herein; and a pharmaceutically acceptable carrier.

The pharmaceutical compositions of the disclosure are formulated with suitable diluents, carriers, excipients, and other agents that provide improved transfer, delivery, tolerance, and the like. The compositions may be formulated for specific uses, such as for veterinary uses or pharmaceutical uses in humans. The form of the composition and the excipients, diluents and/or carriers used will depend upon the intended uses of the antibody and, for therapeutic uses, the mode of administration. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of fusion protein administered to a patient may vary depending upon the age and the size of the patient, target disease, conditions, route of administration, and the like. The preferred dose is typically calculated according to body weight or body surface area. When an antibody of the present disclosure is used for treating a condition or disease associated with PD-L1 in an adult patient, it may be advantageous to intravenously administer the antibody of the present disclosure. Depending on the severity of the condition, the frequency and the duration of the treatment can be adjusted. Effective dosages and schedules for administering the antibody may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition of the present disclosure can be delivered subcutaneously or intravenously with a standard needle and syringe. In addition, with respect to subcutaneous delivery, a pen delivery device readily has disclosures in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Disclosures of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Disclosures of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared. e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Detecting:

The present disclosure further provides a method for detecting a cancer in a subject in need thereof, comprising contacting a sample derived from the subject with the fusion protein as disclosed herein.

The present disclosure further provides a kit for detecting a cancer in a sample, comprising the fusion protein as disclosed herein.

The present disclosure further provides a method for detecting PD-L1 in a sample, comprising contacting the sample with the fusion protein as disclosed herein.

The fusion protein of the present disclosure may also be used to detect and/or measure PD-L1, or PD-L1-expressing cells in a sample, e.g., for diagnostic purposes. For example, an anti-PD-L1 antibody, or the antigen-binding fragment thereof, may be used to diagnose a condition or disease characterized by aberrant expression (e.g., over-expression, under-expression, lack of expression, etc.) of PD-L1. Exemplary diagnostic assays for PD-L1 may comprise, e.g., contacting a sample, obtained from a patient, with an anti-PD-L1 antibody of the disclosure, wherein the anti-PD-L1 antibody is labeled with a detectable label or reporter molecule. Alternatively, an unlabeled anti-PD-L1 antibody can be used in diagnostic disclosures in combination with a secondary antibody which is itself detectably labeled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, beta-galactosidase, horseradish peroxidase, or luciferase. Specific exemplary assays that can be used to detect or measure PD-L1 in a sample include enzyme-linked immunosorbent assay (ELISA), radioimmunoassay (RIA), and fluorescence-activated cell sorting (FACS).

The following examples are provided to aid those skilled in the art in practicing the present disclosure.

### EXAMPLES

The following examples illustrate the development and use of anti-PD-L1×Gas6 binding portion to suppress tumor growth by inducing an “hot tumor” microenvironment.

### Example 1

### [Generation of Recombinant DNA]

Before generating recombinant DNAs, an in silico simulation of the sequences was performed using SnapGene software. Recombinant DNAs combining either anti-PD-L1 or anti-PD-L1 (scFv+Fc) (antigen binding portion) with Gas6 trap (Gas6 binding portion) were constructed in different spatial combinations. The constructions were generated by polymerase chain reaction (PCR)-based cloning and plasmid cloning. The process is briefly described as follows: in PCR-based cloning, insert DNA was amplified by PCR. To digest DNA, PCR products and recipient plasmids were respectively incubated with specific restriction enzymes for at least 4 hours and as long as overnight. Phosphatase was applied to prevent the recipient plasmids from recirculation before running the digested DNA on an agarose gel and performing a gel purification to isolate the DNA in the anticipated size. T4 DNA ligase was used to fuse insert DNA to recipient plasmids. PCR products in the process described above were replaced with donor plasmids in plasmid cloning.

The spatial arrangement and primer sequences used in PCR-based cloning in each format are described below: Format_1 is with Gas6 trap on the C-terminus of anti-PD-L1. Stop codons-lacking anti-PD-L1 is trailed by stop codons-containing Gas6 trap. Primer Seq_1 and primer Seq_2 were used to generate this format.

Gas6 trap was constructed with the Fc of anti-PD-L1 (anti-PD-L1-Fc) to generate Gas6 trap-Fc-based plasmids. The scFv of anti-PD-L1 (anti-PD-L1-scFv) was then introduced into the Gas6 trap-Fc-based plasmids to generate four conformations. Format_2 is with Gas6 trap on the C-terminus of anti-PD-L1-scFv. Anti-PD-L1-scFv was first introduced to the region of anti-PD-L1-Fc where the N-terminus of anti-PD-L1-Fc were coded. Primer Seq_3 was used to generate insert anti-PD-L1-scFc. Gas6 trap was then was tagged behind stop codons-lacking anti-PD-L1-Fc with primer Seq_4 and primer Seq_5.

Conformational optimization was further applied to Format_1, and Format_2. In Format_1, recombinant DNA with anti-PD-L1 trailed by Gas6 trap on the C-terminus of heavy chain constant regions was modified while pTCAE8.3 plasmids were used as recipient plasmids. Sequences coding the heavy chain variable regions of anti-PD-L1 and Gas6 trap in the recombinant DNA were replaced. Primer Seq_6 and primer Seq_7 were used to generate the insert DNA for this process. In Format_2, recombinant DNA with anti-PD-L1-scFv followed subsequently by stop codons-lacking anti-PD-L1-Fc and Gas6 trap was modified while pTCAE8.3 plasmids were used as recipient plasmids. Sequences coding Gas6 trap in this recombinant DNA were replaced. Primer Seq_8 and primer Seq_9 were used to generate the insert DNA for this process.

TABLE 1
The primer sequences of the application

| | Forward | Reverse |
|---|---|---|
| | (SEQ ID NO) | (SEQ ID NO) |
| Primer Seq_1 | gagccacagg | acggatccgc |
| | tgtacactct | caggagacag |
| | (72) | (73) |
| Primer Seq_2 | tgaggatccg | acggatcctc |
| | gcggcggagg | accaccaggg |
| | atct (74) | ccacgaga |
| | | (75) |
| Primer Seq_3 | tccaagcttg | cgaattccct |
| | aggtccagct | cttgatctcc |
| | ggtg (76) | act (77) |
| Primer Seq_4 | tgaattcggt | agcggccgcc |
| | ggaggcggtt | gatccgccac |
| | ca (78) | cgcca (79) |
| Primer Seq_5 | tagcggccgc | tagcggccgc |
| | tgcccccagg | ctaccaccag |
| | ggca | ggccacgaga |
| | (80) | (81) |
| Primer Seq_6 | acccgtcgac | acggatccct |
| | gccaccatgg | actgggggag |
| | (82) | cactgtg |
| | | (83) |
| Primer Seq_7 | acccgtcgac | acggatccct |
| | gccaccatgg | atgtgatggt |
| | (84) | ggctgtgcgg |
| | | ga |
| | | (85) |
| Primer Seq_8 | acccgtcgac | gtgcggccgc |
| | gccaccatgg | ctactggggg |
| | (86) | agcactgt |
| | | (87) |
| Primer Seq_9 | acccgtcgac | gtgcggccgc |
| | gccaccatgg | ctatgtgatg |
| | (88) | gtggctgtg |
| | | (89) |

### [Amplification and Validation of Recombinant DNA]

Recombinant DNAs were mixed with DH5a on ice for 20 minutes before they were transformed into DH5α with 42° C.-incubation for 45 seconds. The transformed DH5α was then incubated at 37° C. for 1 hour in LB medium before it was plated evenly on ampicillin of 50 μg/mL-containing LB agar plates. The LB agar plates were incubated at 37° C. for 16 hours before selecting candidate clones. The candidate clones were then expanded, and their recombinant DNAs were extracted to be examined by performing diagnostic restriction digestion and DNA sequencing.

### [Expression of Abs of Interest]

Mammalian expression systems were used to produce Abs of interest. Briefly, recombinant DNA was transfected into mammalian cells in assistance of chemicals, lipids, or physical engagement. Taking advantage of drug-resistance markers integrated in the recombinant DNA, mammalian cells that took up the recombinant DNA were distinguished from nontransfected cells. To select highly proliferative mammalian cells, wherein the recombinant DNA was integrated, scaling up methotrexate or methionine sulfoxamine selection system was used.

### [Purification of Abs of Interest]

Abs of interest fused with GFP was purified by affinity column chromatography. Briefly, anti-GFP mAb was dialyzed to coupling buffer (0.1 M NaHCO₃, 0.5 M NaCl, pH 8.3), and was then blended with resin in a stoppered vessel. The mixture was afterwards rotated end-over-end for 2 h at room temperature. After the excess anti-GFP mAb was washed away, the remaining active groups was treated by blocking buffer (0.1 M Tris-HCl buffer, pH 8.0). The anti-GFP mAb-containing resin was finally packed to a column. To purify Abs of interest, their lysate supernatant was loaded onto the anti-GFP mAb-coupled affinity chromatography column. The column was then washed with Binding buffer (10 column volumes, PBS, 0.13 M NaCl, 0.01 M Na2HPO4, 0.01 M NaH2PO4, pH 7.4) and eluted with elution buffer (0.1 M glycine-HCl, pH 4.5). Elution fractions were neutralized immediately by addition of a small amount of neutralizing buffer (1 M Tris-HCl. pH 9.0), and were afterwards dialyzed (0.15 M PBS, pH 7.4).

### [Analysis of Abs of Interest]

Purified Abs of interest were analyzed by a discontinuous 12% SDS-PAGE and Coomassie Brilliant Blue R250 stain. In addition, size exclusion chromatography coupled with dynamic light scattering technique was used to detect the aggregation and denaturation of Abs of interest in high resolution. Purification and yields were estimated by the determination of protein content using bio-software Bandscan5.0.

We have examined various spatial conformations. The Gas6 trap can be fused to either the light chain or the heavy chain of the anti-PD-L1 antibody or PD-L1-binding fragment thereof. The Gas6 trap thereof can be fused to either the N-terminus or the C-terminus of a chain of the anti-PD-L1 unit. The anti-PD-L1 unit thereof can either have a light chain and a separate heavy chain, or have a light chain and a heavy chain on a single protein chain (e.g., scFv). We unexpectedly found that the N-terminal fusion construction exhibited relatively lower productivity and significantly reduced biological activity compared to C-terminal fusion construction. The spatial conformations that have favorable yield and biological function in this invention based on the method of trial and error are presented in FIG. 1, wherein the Gas6 trap is fused to the heavy chain C-terminus of the anti-PD-L1 unit comprising either a light chain and a separate heavy chain (Format_1), or a light chain and a heavy chain on a single protein chain (Format_2). Exemplary bifunctional molecules presented in this invention comprise the bifunctional molecules that belong to Format_1 and Format_2. The exemplary bifunctional molecules belonging to Format_1 are example_1, example_3, example_4, example_5 and example_6; and the exemplary bifunctional molecules belonging to Format_2 are example_2, example_7, example_8 and example_9.

In example_1 and example_2, the antibody (j) or antigen-binding fragment thereof comprising a heavy chain of SEQ ID NO: 57 or a substantially similar sequence thereof and a light chain of SEQ ID NO: 58 or a substantially similar sequence thereof. In example_3, example_4 and example_7, the antibody (a) or antigen-binding fragment thereof comprises VH-CDR1 of SEQ ID NO: 3 or a substantially similar sequence thereof; VH-CDR2 of SEQ ID NO: 4 or a substantially similar sequence thereof; VH-CDR3 of SEQ ID NO: 5 or a substantially similar sequence thereof; VL-CDR1 of SEQ ID NO: 10 or a substantially similar sequence thereof; VL-CDR2 of SEQ ID NO: 11 or a substantially similar sequence thereof; and VL-CDR3 of SEQ ID NO: 12 or a substantially similar sequence thereof. In example_5, example_6, example_8 and example_9, the antibody (a) or antigen-binding fragment thereof comprises VH-CDR1 of SEQ ID NO: 96 or a substantially similar sequence thereof; VH-CDR2 of SEQ ID NO: 97 or a substantially similar sequence thereof; VH-CDR3 of SEQ ID NO: 98 or a substantially similar sequence thereof; VL-CDR1 of SEQ ID NO: 99 or a substantially similar sequence thereof; VL-CDR2 of SEQ ID NO: 11 or a substantially similar sequence thereof; and VL-CDR3 of SEQ ID NO: 12 or a substantially similar sequence thereof. In example_3, example_5, example_7 and example_8, the VH of the antibody (a) or antigen-binding fragment thereof comprises a framework represented by the formula: (HC-FR1)-(VH-CDR1)-(HC-FR2)-(VH-CDR2)-(HC-FR3)-(VH-CDR3)-(HC-FR4). In antibody (a) or antigen-binding fragment thereof, the HC-FR1 is SEQ ID NO: 6 or a substantially similar sequence thereof; the HC-FR2 is SEQ ID NO: 7 or a substantially similar sequence thereof; the HC-FR3 is SEQ ID NO: 8 or a substantially similar sequence thereof; and the HC-FR4 is SEQ ID NO: 9 or a substantially similar sequence thereof. The VL of the antibody (a) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4). In antibody (a) or antigen-binding fragment thereof, the LC-FR1 is SEQ ID NO: 13 or a substantially similar sequence thereof; the LC-FR2 is SEQ ID NO: 14 or a substantially similar sequence thereof; the LC-FR3 is SEQ ID NO: 15 or a substantially similar sequence thereof; and the LC-FR4 is SEQ ID NO: 16 or a substantially similar sequence thereof. In example_4, example_6 and example_9, the VH of the antibody (a) or antigen-binding fragment thereof comprises a framework represented by the formula: (HC-FR1)-(VH-CDR1)-(HC-FR2)-(VH-CDR2)-(HC-FR3)-(VH-CDR3)-(HC-FR4). In antibody (a) or antigen-binding fragment thereof, the HC-FR1 is SEQ ID NO: 6 or a substantially similar sequence thereof; the HC-FR2 is SEQ ID NO: 7 or a substantially similar sequence thereof; the HC-FR3 is SEQ ID NO: 8 or a substantially similar sequence thereof; and the HC-FR4 is SEQ ID NO: 9 or a substantially similar sequence thereof. The VL of the antibody (a) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4). In antibody (a) or antigen-binding fragment thereof, the LC-FR1 is SEQ ID NO: 100 or a substantially similar sequence thereof; the LC-FR2 is SEQ ID NO: 101 or a substantially similar sequence thereof; the LC-FR3 is SEQ ID NO: 102 or a substantially similar sequence thereof; and the LC-FR4 is SEQ ID NO: 16 or a substantially similar sequence thereof.

### Example 2

### [PD-L1 Binding Affinity]

The binding abilities of bifunctional molecules and their parental mAbs were examined by FACS and ELISA. Recombinant human PD-L1 proteins mixed with coating solution (Seracare, 5150-0014) in the concentration of 100 ng/well were plated evenly on 96-well plates. The 96-well plates were incubated at 4° C. overnight. On the next day, 0.05% Tween 20-containing phosphate-buffered saline (PBS) was used to wash the plates between steps where different reagents were applied. The plates were blocked with 3% Skim milk-containing PBS at 37° C. for 1 hour after the coating Abs were discarded. Serial diluted Abs of interest in PBS were then applied at 37° C. for 1 hour after the blocking buffer was discarded. Secondary antibody, horseradish peroxidase (HRP)-conjugated goat anti-human IgG antibody, was applied at 37ºC for 1 hour after the Abs of interest were discarded. Finally, color development was accomplished by the oxidation reaction of 3, 3′, 5, 5′-tetramethylbenzidine (TMB). The oxidation reaction of TMB driven by HRP was then stopped by 1N hydrochloride. The optical density (O.D.) on 450/650 nm were read to quantify the PD-L1 binding affinity of bifunctional molecules and parental Abs. The hPD-L1 binding affinity of BfAbs is demonstrated in Table 2, wherein the bifunctional molecule conformations of example_1, example_2, example_3, example_4, example_5, example_6, example_7, example_8 and example_9 demonstrated no obstruction to their hPD-L1 binding affinity.

TABLE 2
The affinity of example_1, example_2, example_3, example_4, example_5,
example_6, example_7, example_8 and example_9 to human PD-L1,
which is comparable to that of their parent antigen-binding fragments,
is listed as follows.
hPD-L1 binding affinity

| | EC₅₀ (×10⁻¹⁰M) |
|---|---|
| atezolizumab | <1 |
| example_1 | <1 |
| example_2 | <1 |
| example_3 | <1 |
| example_4 | <1 |
| example_5 | <1 |
| example_6 | <1 |
| example_7 | <1 |
| example_8 | <1 |
| example_9 | <1 |

### [Gas6 Binding Affinity]

Abs of interest mixed with coating solution in the concentration of 100 ng/well were plated evenly on 96-well plates. The 96-well plates were incubated at 4° C. overnight. On the next day, 0.05% Tween 20-containing phosphate-buffered saline (PBS) was used to wash the plates between steps where different reagents were applied. The plates were blocked with 3% Skim milk-containing PBS at 37° C. for 1 hour after the coating Abs were discarded. Serial diluted human Gas6 recombinant protein (hGas6) in the concentration of 100 μg/well in PBS was then applied at 37° C. for 1 hour after the blocking buffer was discarded. Secondary antibody, HRP-conjugated goat anti-human IgG antibody, was applied at 37ºC for 1 hour after the Gas6 was discarded. Finally, color development was accomplished by the oxidation reaction of TMB. The oxidation reaction of TMB driven by HRP was then stopped by 1N hydrochloride. The O.D. on 450/650 nm were read to quantify the Gas6 binding affinity of bifunctional molecules and reference Abs.

The hGas6 binding affinity of the parental Axl-Fc (works as a Gas6 binding portion, with the C-terminus of Gas6 trap fused to the N-terminus of human Fc) of bifunctional molecules, and MYD1-72 (a reference decoy receptor) is demonstrated in Table 3. The hGas6 binding affinity of bifunctional molecules is demonstrated in Table 4, wherein the bifunctional molecule conformations of example_1, example_2, example_3, example_4, example_5, example_6, example_7, example_8 and example_9 demonstrated no obstruction to their hGas6 binding affinity.

TABLE 3
The affinity of homemade Axl-Fc and reference MYD1-72
(adapted from US8168415B2) to human Gas6 is listed as follows.
hGas6 binding affinity

| | EC₅₀ (×10⁻⁹M) |
|---|---|
| Axl-Fc | <3 |
| MYD1-72 | <3 |

TABLE 4
The affinity of example_1, example_2, example_3, example_4,
example_5, example_6, example_7, example_8 and example_9 to human
Gas6, which is comparable to that of their parent Gas6 binding portions,
is listed as follows.
hGas6 binding affinity

| | EC₅₀ (×10⁻⁹M) |
|---|---|
| example_1 | <3 |
| example_2 | <3 |
| example_3 | <3 |
| example_4 | <3 |
| example_5 | <3 |
| example_6 | <3 |
| example_7 | <3 |
| example_8 | <3 |
| example_9 | <3 |

### Example 3

### [PD-L1/PD-1 Blockade]

A bioassay kit (Promega, J1252) was used to quantify the PD-1/PD-L1 blockade function of bifunctional molecules and reference Abs. Briefly, PD-L1 aAPC/CHO-K1 cells were seeded in the concentration of 4×10⁴/well on 96-well plates. The plates were then incubated at 37° C. overnight. On the next day, serial diluted Abs of interest, and PD-1 effector cells in the concentration of 5×10⁴/well were plated and incubated at 37ºC for 6 hours. The T cell receptor (TCR) and nuclear factor of activated T cells (NFAT)-mediated activation of luciferase activity in the effector cells was finally examined. TCR/NFAT signaling was inhibited by PD-L1/PD-1 signaling. Therefore, the higher the luminescent values of the effector cells, the more effective the Abs were to block PD-L1/PD-1 signaling.

The PD-L1/PD-1 blockade activity of bifunctional molecules is demonstrated in Table 5, wherein the bifunctional molecule conformations of example_1, example_2, example_3, example_4, example_5, example_6, example_7, example_8 and example_9 demonstrated no obstruction to their PD-L1/PD-1 blockade activity.

TABLE 5
example_1, example_2, example_3, example_4, example_5, example_6,
example_7, example_8 and example_9 can efficiently block human
PD-L1/PD-1 signaling.
PD-L1/PD-1 blockade assay

| | EC₅₀ (×10⁻⁹M) |
|---|---|
| atezolizumab | <3 |
| example_1 | <3 |
| example_2 | <3 |
| example_3 | <3 |
| example_4 | <3 |
| example_5 | <3 |
| example_6 | <3 |
| example_7 | <3 |
| example_8 | <3 |
| example_9 | <3 |

### [Gas6/Axl Blockade]

A human Axl-expressing Ba/F3 cell line (Ba/F3-hAxl) was used to quantify the Gas6/Axl blocking function of BfAbs and reference Abs. A mixture consisting Ba/F3-hAxl in the concentration of 2000 cells/well, 10% fetal bovine serum, and hGas6 of 100 pg/well was evenly plated on 96-well plates. Abs of interest in the concentration of 5 ng/well were then added to the plates. The plates were afterwards incubated at 37° C. for 72 hours. The cell viability of Ba/F3-hAxl was examined by CCK-8 reagent (Dojindo, CK04), and the O.D. on 450 nm were finally read.

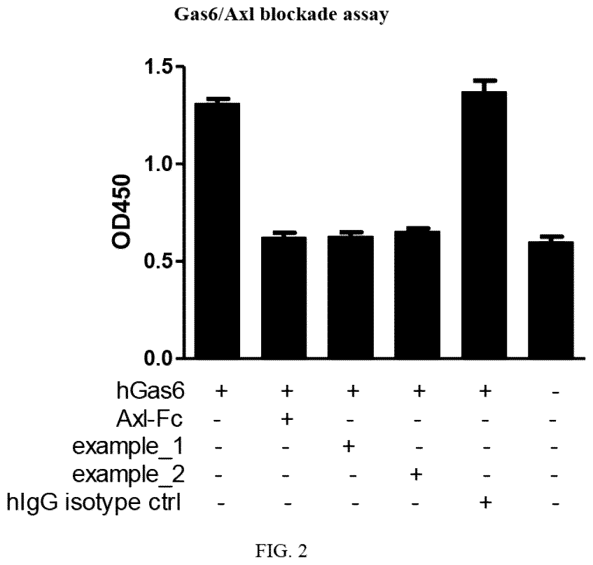

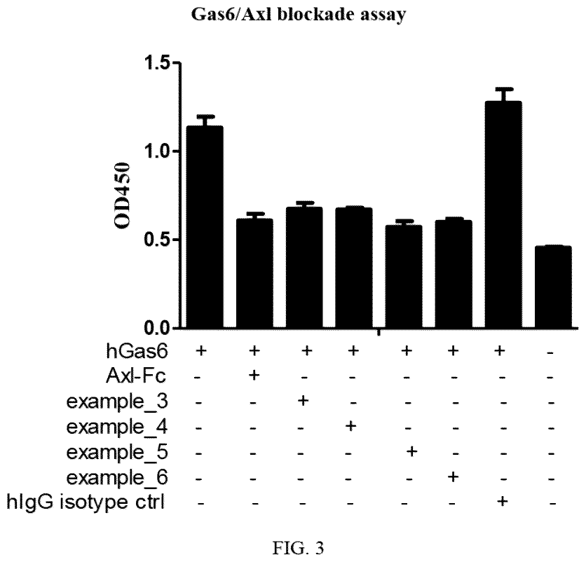

The Gas6/Axl blockade activity of bifunctional molecules and their parental Axl-Fc is demonstrated in FIG. 2, FIG. 3, and FIG. 4, wherein the bifunctional conformations of example_1, example_2, example_3, example_4, example_5, example_6, example_7, example_8, and example_9 demonstrated no obstruction to their Gas6/Axl blockade activity.

### Example 4

### [Cancer Immunotherapy]

EMT-6 (a mouse triple-negative breast cancer cell line that spontaneously secrets Gas6) was inoculated subcutaneously into female BALB/cByJNarl mice. The drug administration was initiated at the mean tumor size of 50-100 mm³. Five dosing groups including hIgG isotype control, atezolizumab, Axl-Fc, atezolizumab in combination with Axl-Fc, and example_2 at dose level of 10 mg/kg were intraperitonealy injected twice a week for three weeks (the actually dosages of treatments were adjusted by their respective molecular weight). Body weight and tumor volumes were measured three to four time a week. Blood samples were collected in 3 h after drug administration. In vivo experiments were reviewed and approved by the Development Center for Biotechnology Laboratories Institutional Animal Care and Use Committee and were done in accordance with the Guide for Care and Use of Laboratory Animals.

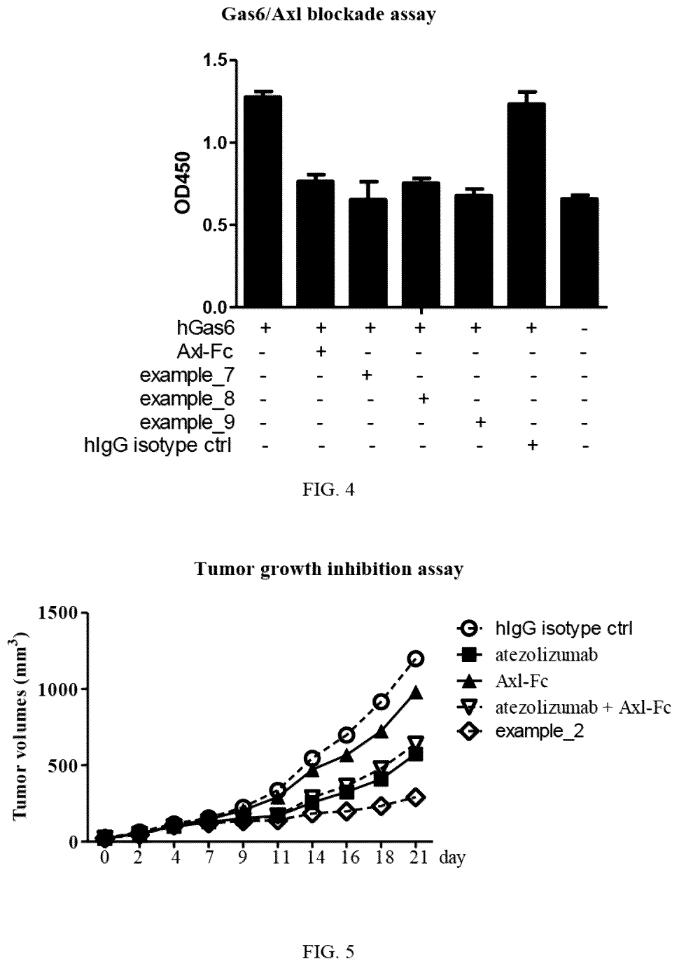

The tumor growth inhibition activity of example_2 and its parental Ab, and Axl-Fc thereof, wherein the parental Ab and Axl-Fc thereof were administered as single agents or in combination, is demonstrated in FIG. 5. As shown in FIG. 5, example_2 is unexpectedly superior to parental Ab and Axl-Fc thereof in tumor growth inhibition activity, wherein the parental Ab and Axl-Fc thereof were administered as single agents or in combination. In parental Ab and the combination of parental Ab and Axl-Fc groups, 3 out of 6 mice bore tumor volume larger than 500 mm³, whereas only 1 out 6 mice bore tumor volume larger than 500 mm³ in example_2 group. Compared to parental Ab, higher complete remission rate was observed in example_2 group, for 1 out of 6 mouse achieved complete remission in parental Ab group while 2 out of 6 mice achieved complete remission in example_2 group.

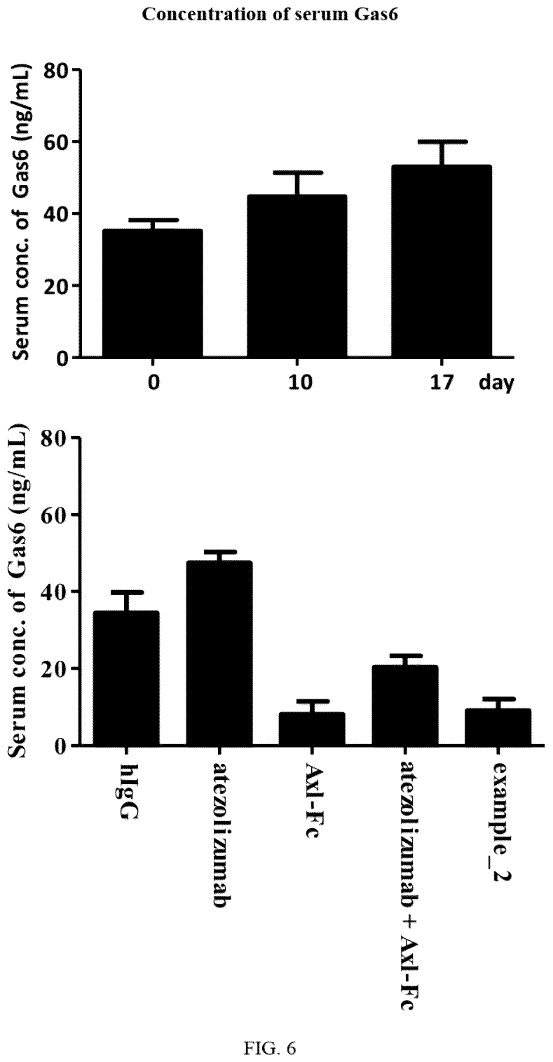

The concentration of serum Gas6 in mice was measured (FIG. 6). The concentration of serum Gas6 in mice decreased once molecules containing the Gas6 binding moiety were administered, demonstrating the impressive in vivo function of the Gas6 binding moiety disclosed in this disclosure.

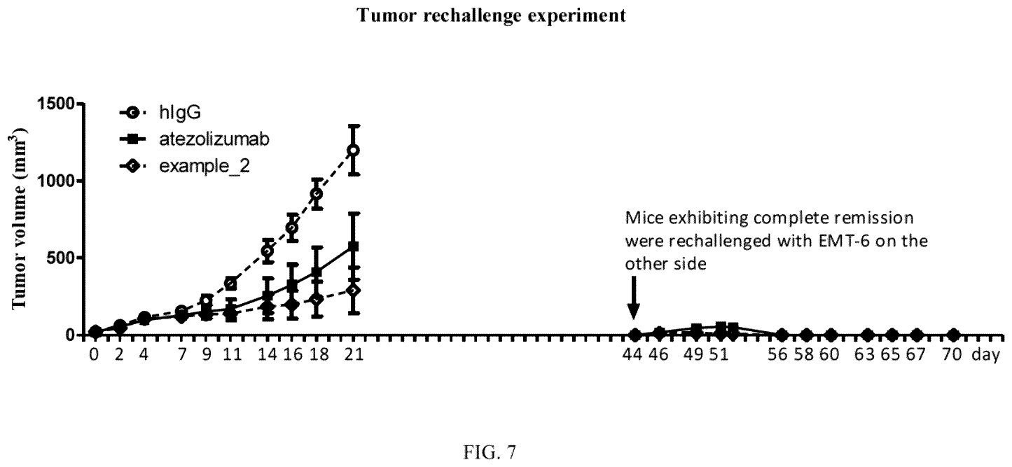

The mice which achieved complete remission were further enrolled in a rechallenge study to examine the immune memory effects of example_2 and its parental Ab. In brief, the same tumor cell line (EMT-6) was inoculated subcutaneously into the mice which achieved complete remission on the first round. The tumor inoculation was performed on the other side of the mice when rechallenge. Body weight and tumor volumes were then measured three to four times a week. The result illustrated in FIG. 7 finally shows that immune memory effects were observed in both example_2 and its parental Ab. Nonetheless, example_2 shows slightly better immune memory responses than does its parental Ab.

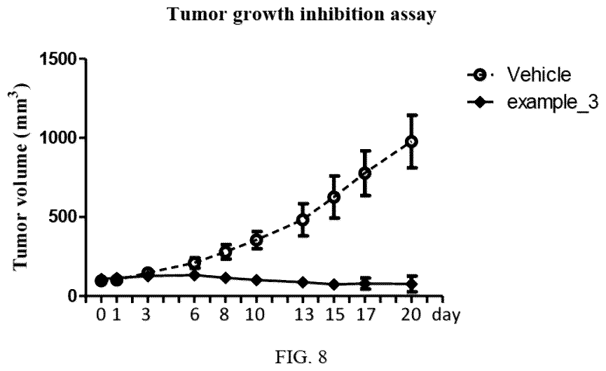

Tumor growth inhibition function of the present invention was confirmed with another embodiment, example_3, in an mPD-L1-hPD-L1+EMT-6 syngeneic mouse model. Compared to the vehicle group, the example_3 group demonstrated the promising anti-cancer effects of the present invention in FIG. 8.

The present disclosure provides a bifunctional fusion protein which has novel compositions, wherein the novel compositions comprise two binding portions: an anti-PD-L1 antibody or a PD-L1 binding fragment fused to a Gas6 binding portion at C-terminus. Different spatial conformations of the bifunctional fusion proteins were examined. Supported by the examples in this disclosure, selected anti-PD-L1×Gas6 trap bifunctional conformations disclosed herein, Format_1 and Format_2, can be applied to anti-PD-L1 antibodies or PD-L1 binding fragments with different sequences. In addition, the anti-PD-L1×Gas6 trap bifunctional fusion proteins in Format_1 or Format_2 showed comparable binding affinity and function to both of their parent binding portions. More importantly, the anti-PD-L1×Gas6 trap bifunctional fusion proteins disclosed herein unexpectedly demonstrated synergistic anti-tumor effects: the selected anti-PD-L1×Gas6 trap bifunctional conformations were superior to parental binding portions in tumor growth inhibition activity, wherein the parental binding portions were administered as single agents or in combination. The superior tumor-targeting effects of the anti-PD-L1×Gas6 trap bifunctional fusion proteins to the combination of the two binding portions as single agents are part of the reasons. Suggested by the differences of the binding affinity between anti-PD-L1 antibodies or PD-L1 binding fragments and Gas6 trap binding portions to their corresponding targets, the anti-PD-L1 antibodies or PD-L1 binding fragments dominated the tumor-targeting effects of the anti-PD-L1×Gas6 trap fusion proteins in this disclosure.

While the present invention has been described in conjunction with the specific embodiments set forth above, many alternatives thereto and modifications and variations thereof will be apparent to those of ordinary skill in the art. All such alternatives, modifications and variations are regarded as falling within the scope of the present invention.

1. A fusion protein comprising
a Gas6 (growth arrest-specific 6) binding portion; and
an antigen binding portion comprising an antibody or antigen-binding fragment thereof that is specific to an epitope in PD-L1 (programmed death-ligand 1).

2. The fusion protein of claim 1, wherein the Gas6 binding portion comprises an extracellular domain of receptor tyrosine kinase (RTK) of TAM (Tyro-3, Axl, MerTK) family.

3. The fusion protein of claim 1, wherein the Gas6 binding portion comprises an amino acid sequence of SEQ ID NO: 1 or 2 or variants thereof.

4. The fusion protein of claim 1, wherein the antibody or antigen-binding fragment thereof comprises complementarity determining regions (CDRs) of a heavy chain variable region (VH) and complementarity determining regions of a light chain variable region (VL), wherein the complementarity determining regions of the heavy chain variable region comprise VH-CDR1, VH-CDR2 and VH-CDR3, and the complementarity determining regions of the light chain variable region comprise VL-CDR1, VL-CDR2 and VL-CDR3, and the antibody or antigen-binding fragment thereof is:
an antibody (a) or antigen-binding fragment thereof comprising VH-CDR1 of SEQ ID NO: 3 or a substantially similar sequence thereof; VH-CDR2 of SEQ ID NO: 4 or a substantially similar sequence thereof; VH-CDR3 of SEQ ID NO: 5 or a substantially similar sequence thereof; VL-CDR1 of SEQ ID NO: 10 or a substantially similar sequence thereof; VL-CDR2 of SEQ ID NO: 11 or a substantially similar sequence thereof; and VL-CDR3 of SEQ ID NO: 12 or a substantially similar sequence thereof, or VH-CDR1 of SEQ ID NO: 96 or a substantially similar sequence thereof; VH-CDR2 of SEQ ID NO: 97 or a substantially similar sequence thereof; VH-CDR3 of SEQ ID NO: 98 or a substantially similar sequence thereof; VL-CDR1 of SEQ ID NO: 99 or a substantially similar sequence thereof; VL-CDR2 of SEQ ID NO: 11 or a substantially similar sequence thereof; and VL-CDR3 of SEQ ID NO: 12 or a substantially similar sequence thereof,
an antibody (b) or antigen-binding fragment thereof comprising VH-CDR1 of SEQ ID NO: 17 or a substantially similar sequence thereof; VH-CDR2 of SEQ ID NO: 18 or a substantially similar sequence thereof; VH-CDR3 of SEQ ID NO: 19 or a substantially similar sequence thereof, VL-CDR1 of SEQ ID NO: 20 or a substantially similar sequence thereof; VL-CDR2 of SEQ ID NO: 21 or a substantially similar sequence thereof; and VL-CDR3 of SEQ ID NO: 22 or a substantially similar sequence thereof,
an antibody (c) or antigen-binding fragment thereof comprising VH-CDR1 of SEQ ID NO: 23 or a substantially similar sequence thereof; VH-CDR2 of SEQ ID NO: 24 or a substantially similar sequence thereof; VH-CDR3 of SEQ ID NO: 25 or a substantially similar sequence thereof, VL-CDR1 of SEQ ID NO: 26 or a substantially similar sequence thereof; VL-CDR2 of SEQ ID NO: 27 or a substantially similar sequence thereof; and VL-CDR3 of SEQ ID NO: 28 or a substantially similar sequence thereof,
an antibody (d) or antigen-binding fragment thereof comprising VH-CDR1 of SEQ ID NO: 29 or a substantially similar sequence thereof; VH-CDR2 of SEQ ID NO: 30 or a substantially similar sequence thereof; VH-CDR3 of SEQ ID NO: 31 or a substantially similar sequence thereof, VL-CDR1 of SEQ ID NO: 32 or a substantially similar sequence thereof; VL-CDR2 of SEQ ID NO: 33 or a substantially similar sequence thereof; and VL-CDR3 of SEQ ID NO: 34 or a substantially similar sequence thereof,
an antibody (e) or antigen-binding fragment thereof comprising VH-CDR1 of SEQ ID NO: 43 or a substantially similar sequence thereof; VH-CDR2 of SEQ ID NO: 44 or a substantially similar sequence thereof; VH-CDR3 of SEQ ID NO: 45 or a substantially similar sequence thereof, VL-CDR1 of SEQ ID NO: 46 or a substantially similar sequence thereof; VL-CDR2 of SEQ ID NO: 47 or a substantially similar sequence thereof; and VL-CDR3 of SEQ ID NO: 48 or a substantially similar sequence thereof,
an antibody (f) or antigen-binding fragment thereof comprising a heavy chain of SEQ ID NO: 49 or a substantially similar sequence thereof and a light chain of SEQ ID NO: 50 or a substantially similar sequence thereof,
an antibody (g) or antigen-binding fragment thereof comprising a heavy chain of SEQ ID NO: 51 or a substantially similar sequence thereof and a light chain of SEQ ID NO: 52 or a substantially similar sequence thereof,
an antibody (h) or antigen-binding fragment thereof comprising a heavy chain of SEQ ID NO: 53 or a substantially similar sequence thereof and a light chain of SEQ ID NO: 54 or a substantially similar sequence thereof,
an antibody (i) or antigen-binding fragment thereof comprising a heavy chain of SEQ ID NO: 55 or a substantially similar sequence thereof and a light chain of SEQ ID NO: 56 or a substantially similar sequence thereof,
an antibody (j) or antigen-binding fragment thereof comprising a heavy chain of SEQ ID NO: 57 or a substantially similar sequence thereof and a light chain of SEQ ID NO: 58 or a substantially similar sequence thereof, or
an antibody (k) or antigen-binding fragment thereof comprising a heavy chain encoded by SEQ ID NO: 59 or a substantially similar sequence thereof; and a light chain encoded by SEQ ID NO: 60 or a substantially similar sequence thereof.

5. The fusion protein of claim 4, wherein the VH of the antibody (a) or antigen-binding fragment thereof comprises a framework represented by the formula:
(HC-FR1)-(VH-CDR1)-(HC-FR2)-(VH-CDR2)-(HC-FR3)-(VH-CDR3)-(HC-FR4); the HC-FR1 is SEQ ID NO: 6 or a substantially similar sequence thereof; the HC-FR2 is SEQ ID NO: 7 or a substantially similar sequence thereof; the HC-FR3 is SEQ ID NO: 8 or a substantially similar sequence thereof; and the HC-FR4 is SEQ ID NO: 9 or a substantially similar sequence thereof; and the VL of the antibody (a) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4); the LC-FR1 is SEQ ID NO: 13 or a substantially similar sequence thereof; the LC-FR2 is SEQ ID NO: 14 or a substantially similar sequence thereof; the LC-FR3 is SEQ ID NO: 15 or a substantially similar sequence thereof; and the LC-FR4 is SEQ ID NO: 16 or a substantially similar sequence thereof; or (HC-FR1)-(VH-CDR1)-(HC-FR2)-(VH-CDR2)-(HC-FR3)-(VH-CDR3)-(HC-FR4); the HC-FR1 is SEQ ID NO: 6 or a substantially similar sequence thereof; the HC-FR2 is SEQ ID NO: 7 or a substantially similar sequence thereof; the HC-FR3 is SEQ ID NO: 8 or a substantially similar sequence thereof; and the HC-FR4 is SEQ ID NO: 9 or a substantially similar sequence thereof; and the VL of the antibody (a) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4); the LC-FR1 is SEQ ID NO: 100 or a substantially similar sequence thereof; the LC-FR2 is SEQ ID NO: 101 or a substantially similar sequence thereof; the LC-FR3 is SEQ ID NO: 102 or a substantially similar sequence thereof; and the LC-FR4 is SEQ ID NO: 16 or a substantially similar sequence thereof.

6. The fusion protein of claim 4, wherein the VH of the antibody (d) or antigen-binding fragment thereof comprises a framework represented by the formula: (HC-FR1)-(VH-CDR1)-(HC-FR2)-(VH-CDR2)-(HC-FR3)-(VH-CDR3)-(HC-FR4); the HC-FR1 is SEQ ID NO: 35 or a substantially similar sequence thereof; the HC-FR2 is SEQ ID NO: 36 or a substantially similar sequence thereof; the HC-FR3 is SEQ ID NO: 37 or a substantially similar sequence thereof; and the HC-FR4 is SEQ ID NO: 38 or a substantially similar sequence thereof; and the VL of the antibody (d) or antigen-binding fragment thereof comprises a framework represented by the formula: (LC-FR1)-(VL-CDR1)-(LC-FR2)-(VL-CDR2)-(LC-FR3)-(VL-CDR3)-(LC-FR4); the LC-FR1 is SEQ ID NO: 39 or a substantially similar sequence thereof; the LC-FR2 is SEQ ID NO: 40 or a substantially similar sequence thereof; the LC-FR3 is SEQ ID NO: 41 or a substantially similar sequence thereof; and the LC-FR4 is SEQ ID NO: 42 or a substantially similar sequence thereof.

7. The fusion protein of claim 1, wherein the antibody or antigen-binding fragment thereof comprises a heavy chain constant region and a light chain constant region, and the constant region contains a mutation at an amino acid position corresponding to N297 of IgG1.

8. The fusion protein of claim 1, wherein the antigen binding portion comprises an Fab fragment, an F(ab′)₂ fragment, an ScFv fragment, a chimeric antibody, or a nanobody.

9. The fusion protein of claim 1, wherein the Gas6 binding portion is fused to the antigen binding portion through a peptide linker.

10. The fusion protein of claim 9, wherein the peptide linker comprises an amino acid sequence of SEQ ID NO: 67, SEQ ID NO: 68, SEQ ID NO: 69, SEQ ID NO: 70 or SEQ ID NO: 71.

11. The fusion protein of claim 1, wherein the Gas6 binding portion is fused to a heavy chain of the antigen binding portion.

12. The fusion protein of claim 1, wherein the Gas6 binding portion is fused to the C-terminus of a heavy chain of the antigen binding portion.

13. A method for treating, prophylactic treating and/or preventing a cancer in a subject in need thereof, comprising administering the subject with an effective amount of the fusion protein of claim 1.

14. The method of claim 13, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer, and thyroid cancer.

15. A method for detecting a cancer in a subject in need thereof, comprising contacting a sample derived from the subject with the fusion protein of claim 1.

16. The method of claim 15, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer, and thyroid cancer.

17. A kit for detecting a cancer in a sample, comprising the fusion protein of claim 1.

18. The kit of claim 17, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer, and thyroid cancer.

19. A method for detecting PD-L1 in a sample, comprising contacting the sample with the fusion protein of claim 1.

20. A method for neutralizing Gas6 in a sample, comprising contacting the sample with the fusion protein of claim 1.


SEQUENCE LISTING (118 sequences)

SEQ ID NO: 1 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 197 aa, Homo sapiens
APRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATIT

SEQ ID NO: 3 - protein, 10 aa, synthetic construct
GYSITSDYWN

SEQ ID NO: 4 - protein, 16 aa, synthetic construct
YISYTGSTYYNPSLKS

SEQ ID NO: 5 - protein, 10 aa, synthetic construct
RGEWLSPFAY

SEQ ID NO: 6 - protein, 25 aa, synthetic construct
EVQLQESGPGLVKPSQTLSLTCTVS

SEQ ID NO: 7 - protein, 14 aa, synthetic construct
WIRKPPGKGLEYMG

SEQ ID NO: 8 - protein, 32 aa, synthetic construct
RITISRDTSKNQYSLKLSSVTAADTAVYYCAR

SEQ ID NO: 9 - protein, 11 aa, synthetic construct
WGQGTLVTVSS

SEQ ID NO: 10 - protein, 17 aa, synthetic construct
KSSQSLLYSSNQKNSLA

SEQ ID NO: 11 - protein, 7 aa, synthetic construct
WASTRES

SEQ ID NO: 12 - protein, 9 aa, synthetic construct
QQYYTYPFT

SEQ ID NO: 13 - protein, 23 aa, synthetic construct
DIQMTQSPSSLSASVGDRVTITC

SEQ ID NO: 14 - protein, 15 aa, synthetic construct
WYQQKPGKAPKLLIY

SEQ ID NO: 15 - protein, 32 aa, synthetic construct
GVPSRFSGSGSGTDFTLTISSLQPEDFATYYC

SEQ ID NO: 16 - protein, 10 aa, synthetic construct
FGQGTKLEIK

SEQ ID NO: 17 - protein, 5 aa, synthetic construct
SYIMM

SEQ ID NO: 18 - protein, 17 aa, synthetic construct
SIYPSGGITFYADTVKG

SEQ ID NO: 19 - protein, 11 aa, synthetic construct
IKLGTVTTVDY

SEQ ID NO: 20 - protein, 14 aa, synthetic construct
TGTSSDVGGYNYVS

SEQ ID NO: 21 - protein, 7 aa, synthetic construct
DVSNRPS

SEQ ID NO: 22 - protein, 10 aa, synthetic construct
SSYTSSSTRV

SEQ ID NO: 23 - protein, 5 aa, synthetic construct
MYMMM

SEQ ID NO: 24 - protein, 17 aa, synthetic construct
SIYPSGGITFYADSVKG

SEQ ID NO: 25 - protein, 11 aa, synthetic construct
IKLGTVTTVDY

SEQ ID NO: 26 - protein, 14 aa, synthetic construct
TGTSSDVGAYNYVS

SEQ ID NO: 27 - protein, 7 aa, synthetic construct
DVSNRPS

SEQ ID NO: 28 - protein, 10 aa, synthetic construct
SSYTSSSTRV

SEQ ID NO: 29 - protein, 5 aa, synthetic construct
SYIMM

SEQ ID NO: 30 - protein, 17 aa, synthetic construct
SIYPSGGITFYAPTVKG

SEQ ID NO: 31 - protein, 11 aa, synthetic construct
IKLGTVTTVDY

SEQ ID NO: 32 - protein, 14 aa, synthetic construct
TGTSSDVGGYNYVS

SEQ ID NO: 33 - protein, 7 aa, synthetic construct
DVSNRPS

SEQ ID NO: 34 - protein, 10 aa, synthetic construct
SSYTSSSTRV

SEQ ID NO: 35 - protein, 30 aa, synthetic construct
EVQLLESGGGLVQPGGSLRLSCAASGFTGS

SEQ ID NO: 36 - protein, 14 aa, synthetic construct
WVRQAPGKGLEWVS

SEQ ID NO: 37 - protein, 32 aa, synthetic construct
RFTISRDNSKNTLYLQMNSLRAEDTAVYYCAR

SEQ ID NO: 38 - protein, 11 aa, synthetic construct
WGQGTLVTVSS

SEQ ID NO: 39 - protein, 22 aa, synthetic construct
QSALTQPASVSGSPGQSITISC

SEQ ID NO: 40 - protein, 15 aa, synthetic construct
WYQQHPGKAPKLMIY

SEQ ID NO: 41 - protein, 32 aa, synthetic construct
GVSNRFSGSKSGNTASLTISGLQAEDEADYYC

SEQ ID NO: 42 - protein, 10 aa, synthetic construct
FGTGTKVTVL

SEQ ID NO: 43 - protein, 5 aa, synthetic construct
SYIMM

SEQ ID NO: 44 - protein, 17 aa, synthetic construct
SIYPSGGITGYADTVKG

SEQ ID NO: 45 - protein, 11 aa, synthetic construct
IKLGTVTTVDY

SEQ ID NO: 46 - protein, 14 aa, synthetic construct
TGTSSDVGGYNYVS

SEQ ID NO: 47 - protein, 7 aa, synthetic construct
DVSNRPS

SEQ ID NO: 48 - protein, 10 aa, synthetic construct
SSYTSSSTRV

SEQ ID NO: 49 - protein, 119 aa, synthetic construct
EVQLLESGGGLVQPGGSLRLSCAASGFTFSSYIMMVWRQAPGKGLEWVSSIYPSGGITFYADWKGRFTISRDNSKNTLYLQMNSLRAEDTAVYYCARIKLGTVTTVDYWGQGTLVYVSS

SEQ ID NO: 50 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLOAEDEADYYCSSYTSSSTRVFGTGTKVTVL

SEQ ID NO: 51 - protein, 120 aa, synthetic construct
EVQLLESGGGLVQPGGSLRLSCAASGFTFSMYMMMWVRQAPGKGLEVWSSIYPSGGITFYADSVKGRFTISRDNSKNTLYLQMNSLRAEDTAIYYCARIKLGTVTTVDYWGQGTLVTVSS

SEQ ID NO: 52 - protein, 108 aa, synthetic construct
QSALTQPASVSPGQSITISCTGTSSDVGAYNYVSWYQQHPGKAPKLMIYDVSNRPSGVSNRFSGSKSGNTASLTISGLQAEDEADYYCSSYTSSSTRVFGTGTKVTVL

SEQ ID NO: 53 - protein, 118 aa, synthetic construct [sequence omitted]

SEQ ID NO: 54 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 55 - protein, 118 aa, synthetic construct [sequence omitted]

SEQ ID NO: 56 - protein, 108 aa, synthetic construct; identical to SEQ ID NO: 54

SEQ ID NO: 57 - protein, 118 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 58 - protein, 108 aa, synthetic construct; identical to SEQ ID NO: 54

SEQ ID NO: 59 - DNA, 1407 bp, synthetic construct
  source 1..1407: mol_type = other DNA
atggagttgcctgttaggctgttggtgctgatgttctggattcctgctagctccagcgaggtgcagctgctggaatccggcggaggactggtgcagcctggcggctccctgagactgtcttgcgccgcctccggcttcaccttctccagctacatcatgatgtgggtgcgacaggcccctggcaagggcctggaatgggtgtcctccatctacccctccggcggcatcaccttctacgccgacaccgtgaagggccggttcaccatctcccgggacaactccaagaacaccctgtacctgcagatgaactccctgcgggccgaggacaccgccgtgtactactgcgcccggatcaagctgggcaccgtgaccaccgtggactactggggccagggcaccctggtgacagtgtcctccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 60 - DNA, 705 bp, synthetic construct
  source 1..705: mol_type = other DNA
atggagttgcctgttaggctgttggtgctgatgttctggattcctgcttccttaagccagtccgccctgacccagcctgcctccgtgtctggctcccctggccagtccatcaccatcagctgcaccggcacctccagcgacgtgggcggctacaactacgtgtcctggtatcagcagcaccccggcaaggcccccaagctgatgatctacgacgtgtccaaccggccctccggcgtgtccaacagattctccggctccaagtccggcaacaccgcctccctgaccatcagcggactgcaggcagaggacgaggccgactactactgctcctcctacacctcctccagcaccagagtgttcggcaccggcacaaaagtgaccgtgctgggccagcccaaggccaacccaaccgtgacactgttccccccatcctccgaggaactgcaggccaacaaggccaccctggtctgcctgatctcagatttctatccaggcgccgtgaccgtggcctggaaggctgatggctccccagtgaaggccggcgtggaaaccaccaagccctccaagcagtccaacaacaaatacgccgcctcctcctacctgtccctgacccccgagcagtggaagtcccaccggtcctacagctgccaggtcacacacgagggctccaccgtggaaaagaccgtcgcccccaccgagtgctca

SEQ ID NO: 61 - protein, 101 aa, Homo sapiens
PRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFV

SEQ ID NO: 62 - protein, 84 aa, Homo sapiens
PYFLEEPEDRTVAANTPFNLSCQAQGPPEPVDLLWLQDAVPLATAPGHGPQRSLHVPGLNKTSSFSCEAHNAKGVTTSRTATIT

SEQ ID NO: 63 - protein, 108 aa, Homo sapiens
PQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGV

SEQ ID NO: 64 - protein, 95 aa, Homo sapiens
PLGPPENISATRNGSQAFVHWQEPRAPLQGTLLGYRLAYQGQDTPEVLMDIGLRQEVTLELQGDGSVSNLTVCVAAYTAAGDGPWSLPVPLEAWR

SEQ ID NO: 65 - protein, 422 aa, Homo sapiens
HRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 66 - protein, 15 aa, Homo sapiens
LVKEPSTPAFSWPWW

SEQ ID NO: 67 - protein, 25 aa, synthetic construct
IPPHVQKSVNNDMIVTDNNGAVKFP

SEQ ID NO: 68 - protein, 5 aa, synthetic construct
GGGGS

SEQ ID NO: 69 - protein, 25 aa, synthetic construct
  VARIANT 7
  VARIANT 8
  VARIANT 19
IPPHVQJJVNNDMIVTDNJGAVKFP

SEQ ID NO: 70 - protein, 25 aa, synthetic construct
TAGHTQTSTGGGAITTGTSGAGHGP

SEQ ID NO: 71 - protein, 9 aa, synthetic construct
EEYNTSNPD

SEQ ID NO: 72 - DNA, 20 bp, synthetic construct
  source 1..20: mol_type = other DNA
gagccacaggtgtacactct

SEQ ID NO: 73 - DNA, 20 bp, synthetic construct
  source 1..20: mol_type = other DNA
acggatccgccaggagacag

SEQ ID NO: 74 - DNA, 24 bp, synthetic construct
  source 1..24: mol_type = other DNA
tgaggatccggcggcggaggatct

SEQ ID NO: 75 - DNA, 28 bp, synthetic construct
  source 1..28: mol_type = other DNA
acggatcctcaccaccagggccacgaga

SEQ ID NO: 76 - DNA, 24 bp, synthetic construct
  source 1..24: mol_type = other DNA
tccaagcttgaggtccagctggtg

SEQ ID NO: 77 - DNA, 23 bp, synthetic construct
  source 1..23: mol_type = other DNA
cgaattccctcttgatctccact

SEQ ID NO: 78 - DNA, 22 bp, synthetic construct
  source 1..22: mol_type = other DNA
tgaattcggtggaggcggttca

SEQ ID NO: 79 - DNA, 25 bp, synthetic construct
  source 1..25: mol_type = other DNA
agcggccgccgatccgccaccgcca

SEQ ID NO: 80 - DNA, 24 bp, synthetic construct
  source 1..24: mol_type = other DNA
tagcggccgctgcccccaggggca

SEQ ID NO: 81 - DNA, 30 bp, synthetic construct
  source 1..30: mol_type = other DNA
tagcggccgcctaccaccagggccacgaga

SEQ ID NO: 82 - DNA, 20 bp, synthetic construct
  source 1..20: mol_type = other DNA
acccgtcgacgccaccatgg

SEQ ID NO: 83 - DNA, 27 bp, synthetic construct
  source 1..27: mol_type = other DNA
acggatccctactgggggagcactgtg

SEQ ID NO: 84 - DNA, 20 bp, synthetic construct
  source 1..20: mol_type = other DNA
acccgtcgacgccaccatgg

SEQ ID NO: 85 - DNA, 32 bp, synthetic construct
  source 1..32: mol_type = other DNA
acggatccctatgtgatggtggctgtgcggga

SEQ ID NO: 86 - DNA, 20 bp, synthetic construct
  source 1..20: mol_type = other DNA
acccgtcgacgccaccatgg

SEQ ID NO: 87 - DNA, 28 bp, synthetic construct
  source 1..28: mol_type = other DNA
gtgcggccgcctactgggggagcactgt

SEQ ID NO: 88 - DNA, 20 bp, synthetic construct
  source 1..20: mol_type = other DNA
acccgtcgacgccaccatgg

SEQ ID NO: 89 - DNA, 29 bp, synthetic construct
  source 1..29: mol_type = other DNA
gtgcggccgcctatgtgatggtggctgtg

SEQ ID NO: 90 - protein, 20 aa, Homo sapiens
LRMFSGTPVIRLRFKRLQPT

SEQ ID NO: 91 - protein, 11 aa, Homo sapiens
EIVGRVTSSGP

SEQ ID NO: 92 - protein, 8 aa, Homo sapiens
RNLVIKVN

SEQ ID NO: 93 - protein, 9 aa, Homo sapiens
DAVMKIAVA

SEQ ID NO: 94 - protein, 16 aa, Homo sapiens
ERGLYHLNLTVGIPFH

SEQ ID NO: 95 - protein, 17 aa, Homo sapiens
WLNGEDTTIQETVVNRM

SEQ ID NO: 96 - protein, 10 aa, synthetic construct
GYSITSDYWD

SEQ ID NO: 97 - protein, 16 aa, synthetic construct
YISYTGSTYYNPSLRS

SEQ ID NO: 98 - protein, 10 aa, synthetic construct
RGGWLSPFVY

SEQ ID NO: 99 - protein, 17 aa, synthetic construct
KSRQSLLFSSNQKNSLA

SEQ ID NO: 100 - protein, 23 aa, synthetic construct
DIVMTQSPDSLAVSLGERATINC

SEQ ID NO: 101 - protein, 15 aa, synthetic construct
WYQQKPGQPPKLLIY

SEQ ID NO: 102 - protein, 32 aa, synthetic construct
GVPDRFSGSGSGTDFTLTISSLQAEDVAVYYC

SEQ ID NO: 103 - protein, 25 aa, synthetic construct
QVQLQESGPGLAKPSQTLSLTCSVT

SEQ ID NO: 104 - protein, 14 aa, synthetic construct
WIRKFPGNKLEFMG

SEQ ID NO: 105 - protein, 32 aa, synthetic construct
RISITRDTSKNQYYLQLNSVTTEDTATYYCAR

SEQ ID NO: 106 - protein, 11 aa, synthetic construct
WGQGTLVTVSA

SEQ ID NO: 107 - protein, 23 aa, synthetic construct
DIVMSQSPSSLGVSVGEKITMSC

SEQ ID NO: 108 - protein, 15 aa, synthetic construct
WYQQKPGQSPKLLIY

SEQ ID NO: 109 - protein, 32 aa, synthetic construct
GVPDRFTGSGSGTDFTLTISSVKSEDLAVYYC

SEQ ID NO: 110 - protein, 10 aa, synthetic construct
FGAGTNLELK

SEQ ID NO: 111 - protein, 25 aa, synthetic construct
EVQLQESGPGLTKPSQTLSLTCSVT

SEQ ID NO: 112 - protein, 14 aa, synthetic construct
WIRKFPGNKLEYMG

SEQ ID NO: 113 - protein, 32 aa, synthetic construct
RISITRDTSKNQYYLQLSSVTSEDSATYYCAR

SEQ ID NO: 114 - protein, 11 aa, synthetic construct
WGQGTLVTVSA

SEQ ID NO: 115 - protein, 23 aa, synthetic construct
DTVMSQSPSSLGVSVGERVTLTC

SEQ ID NO: 116 - protein, 15 aa, synthetic construct
WYQQKPGQSPKLLIY

SEQ ID NO: 117 - protein, 32 aa, synthetic construct; identical to SEQ ID NO: 109

SEQ ID NO: 118 - protein, 10 aa, synthetic construct
FGAGTSLELK